(19) United States
(12) Patent Application Publication
(10) Pub. No.: US 2006/0105995 A1
(43) Pub. Date: May 18, 2006

(54) TAFI inhibitors and their use to treat pulmonary fibrosis

(75) Inventors: Hajime Fujimoto, Tsu City (JP); Esteban Cesar Gabazza, Tsu City (JP); Michael John Morser, San Francisco, CA (US); Mariko Nagashima, Belmont, CA (US); Osamu Taguchi, Tsu City (JP)

Correspondence Address: BERLEX BIOSCIENCES;PATENT DEPARTMENT, 2600 HILLTOP DRIVE, P.O. BOX 4099, RICHMOND, CA, 94804-0099, US

(73) Assignees: Schering Aktiengesellschaft, Berlin (DE); Mie University Graduate School of Medicine, Tsu City (JP)

(21) Appl. No.: 11/242,524

(22) Filed: Oct. 3, 2005

Related U.S. Application Data
(60) Provisional application No. 60/616,284, filed on Oct. 5, 2004.

(51) Int. Cl. A61K 31/69; A61K 31/445; A61K 31/41; A61K 31/66; A61K 31/195

(52) U.S. Cl. 514/64; 514/114; 514/381; 514/315; 514/562

(57) ABSTRACT

The invention relates to TAFI inhibitors and their use to treat pulmonary fibrosis.

This application claims priority to U.S. Provisional Application Ser. No. 60/616,284, filed Oct. 5, 2004, the entirety of which is incorporated herein by reference.

### FIELD OF THE INVENTION

The invention relates to TAFI inhibitors and their use to treat pulmonary fibrosis.

### BACKGROUND OF THE INVENTION

Lung fibrosis is the end-stage of a heterogeneous group of respiratory disorders that results from injury to the lung parenchyma, increased proliferation of mesenchymal cells and excessive accumulation of extracellular matrix in the lung (Canonico, A. E., and Brigham, K. L., “Biology of acute lung injury”, In R. G. Crystal, P. J. Barnes, J. B. West, and E. R. Weibel, eds, THE LUNG, 2^{nd} edition, Lippincott-Raven, Philadelphia, pp. 2475-2498 (1997)). There are several causes of pulmonary fibrosis. Interstitial lung diseases (ILD) may be associated with collagen vascular diseases (collagen vascular disease-associated ILD), radiation pneumonitis, pneumoconiosis or sarcoidosis. But most cases of lung fibrosis are of unknown cause, and they are categorized as idiopathic pulmonary fibrosis (IPF). Also, acute lung injury such as that occurring in the acute respiratory distress syndrome (ARDS) may also result in lung fibrosis.

Decreased degradation of extracellular matrix due to deficient function of alveolar fibrinolysis may play a fundamental role in driving the fibrotic response in the lung (Chambers R. C., “Role of coagulation cascade proteases in lung repair and fibrosis”, Eur. Respir. J. Suppl; 44:33s-35s (2003); Swaisgood, C. M. et al. “The development of bleomycin-induced pulmonary fibrosis in mice deficient for components of the fibrinolytic system”, Am. J. Pathol. 157:177-87(2000)). The effector enzyme of the fibrinolytic system is plasmin, which results from the activation of plasminogen by urokinase (uPA) or tissue plasminogen activator (tPA). Plasmin promotes extracellular matrix degradation by directly degrading a number of extracellular matrix macromolecules, or by activating several pro-metalloproteinases and pro-stromelysins (Saksela, O., Rifkin, D. B., “Cell-associated plasminogen activation: regulation and physiological functions”, Ann. Rev. Cell Biol. 4: 93-126 (1988)). Plasmin can also rapidly degrade fibrin formed after leakage of proteins into the alveolar space and activation of the coagulation cascade (Saksela et al., ibid.). Under physiological conditions, the alveolar space of the lung has potent fibrinolytic activity. However, patients with lung injury such as acute respiratory distress syndrome and interstitial lung diseases have low alveolar fibrinolytic function (Bertozzi, P., et al., “Depressed bronchoalveolar urokinase activity in patients with adult respiratory distress syndrome”, N. Engl. J. Med. 322:890-897 (1990)). Animal models of lung injury such as that induced by bleomycin or lipopolysaccharide also show deficient activation of the intraalveolar plasminogen-plasmin system (Idell, S., et al., “Local abnormalities in coagulation and fibrinolytic pathways predispose to alveolar fibrin deposition in the adult respiratory distress syndrome”, J. Clin. Invest. 84:695-705 (1989)). The protective role of plasmin in lung fibrosis has been recently demonstrated in experiments using animals genetically engineered to express null or high concentration of plasminogen activator inhibitor (PAI)-1, the main inhibitor of plasmin generation (Hattori, N., et al., “Bleomycin-induced pulmonary fibrosis in fibrinogen-null mice”, J Clin Invest. 106:1341-50 (2000)). The results of these studies have shown that bleomycin-induced lung fibrosis is more severe in transgenic mice over-expressing PAI-1 than in PAI-1-deficient mice, and bleomycin-treated PAI-1 deficient mice present less lung fibrosis and better outcomes than mice with PAI-1 over-expression (Hattori et al., ibid). Inhibition of plasmin in PAI-1-deficient mice following treatment with bleomycin also caused an increased fibrin and collagen deposition in the lung (Loskutoff, D. J., Quigley, J. P., “PAI-1, fibrosis, and the elusive provisional fibrin matrix”, J. Clin. Invest. 106: 1441-1443 (2000)).

The cause of the low fibrinolytic function in lung injury is not clear but PAI-1 may be responsible. PAI-1 is a member of the serine protease inhibitor gene family that rapidly and potently inhibits both uPA and tPA (Saksela et al., supra). Mice that are deficient in PAI-1 display enhanced fibrinolytic activity. The bronchoalveolar lavage fluid (BALF) from patients with acute respiratory distress syndrome and idiopathic pulmonary fibrosis has dramatically high concentrations of PAI-1, and this has been shown to reduce the fibrinolytic activity of the fluid (Idell et al. supra). Similar findings have been reported in animal models of lung injury induced by bleomycin or lipopolysaccarides (Yasui, H., et al., “Intratracheal administration of activated protein C inhibits bleomycin-induced lung fibrosis in the mouse” Am. J. Respir. Crit. Care Med. 163:1660-1668 (2001); Shimizu, S., et al., “Activated protein C inhibits the expression of platelet-derived growth factor in the lung”, Am. J. Respir. Crit. Care Med. 167:1416-26 (2003)).

Another candidate that may explain the decreased plasmin generation in lung injury is thrombin-activatable fibrinolysis inhibitor (TAFI). TAFI is a glycoprotein with a molecular weight of 55 kDa. TAFI is secreted as a zymogen and it is activated by thrombin-, thrombin-thrombomodulin complex-, plasmin- or trypsin-catalyzed proteolysis to activated TAFI (TAFIa)—a carboxypeptidase that inhibits fibrinolysis (Bajzar, L., “Thrombin activatable fibrinolysis inhibitor and an antifibrinolytic pathway”, Arterioscler. Thromb. Vasc. Biol. 20:2511-2518 (2000)). Activated TAFI reduces generation of plasmin because it removes the carboxy-terminal lysine residues from partially degraded fibrin and thereby abrogates the fibrin cofactor function in the tPA-mediated catalysis of plasminogen to plasmin. Activated TAFI may also directly inactivate plasmin further impairing fibrinolysis (Bajzar, ibid). Although most of data on the function of TAFI in the regulation of fibrinolysis is derived from in vitro studies, recent studies have shown that this function of TAFI is also of fundamental importance in vivo. For example, inhibition of TAFI accelerated thrombolysis in a rabbit model of jugular vein thrombolysis, and increased fibrinolysis and enhanced thioglycollate-induced leukocyte recruitment have been demonstrated in TAFI deficient mice (Minnema, M. C., et al., “Enhancement of rabbit jugular vein thrombolysis by neutralization of factor XI. In vivo evidence for a role of factor XI as an anti-fibrinolytic factor”, J. Clin. Invest. 101:1 0-4 (1998); Swaisgood, C. M., et al., “In vivo regulation of plasminogen function by plasma carboxypeptidase B”, J Clin Invest. 110:1275-82 (2000)).

Clinical studies suggested that TAFI also plays a role in the impairment of fibrinolytic function in lung injury. Patients with lung injury, including those with idiopathic pulmonary fibrosis, have an increased intraalveolar level of TAFI, and this abnormality has been linked to decreased urokinase activity in the lung (Fujimoto, H., et al., “Thrombin-activatable fibrinolysis inhibitor and protein C inhibitor in interstitial lung disease” Am. J Respir. Crit. Care Med. 167:1687-94 (2003)). This observation implicates TAFI in the fibrinolytic dysfunction of lung injury. In addition, the high concentration of TAFI in patients with lung injury has been found to be significantly associated with activation of coagulation system and with markers of inflammation and collagen deposition in the lung, further suggesting the role of TAFI in the pathogenesis of pulmonary fibrosis (Fujimoto et al., ibid).

TAFI inhibors are known in the art, and include compounds such as those disclosed in WO 03/080631, WO 03/13526, WO 00/066550, WO 00/066557, WO 03/027128, WO 01/19836 and WO 02/14285. The entirety of each of these publications disclosing TAFI inhibitors is incorporated herein by reference. Known TAFI inihibitors further include AZD-9684 (Astra Zeneca) and EF-6265 (Meiji Seika Kaisha).

### SUMMARY OF THE INVENTION

Decreased fibrinolytic function favors the development of pulmonary fibrosis. Thrombin-activatable fibrinolysis inhibitor (TAFI) is a strong suppressor of fibrinolysis but its role in lung fibrosis is unknown. To clarify this, we compared bleomycin-induced lung fibrosis in TAFI (−/−), TAFI (+/−) and TAFI (+/+) mice. The results of our studies suggest that the anti-fibrinolytic activity of TAFI contributes to the development of lung fibrosis. Accordingly, TAFI inhibitors would be expected to constitute a possible treatment for pulmonary fibrosis. Preferred TAFI inhibitors include compounds such as those disclosed in WO 03/080631, the entirety of which is incorporated herein by reference.

Accordingly, in one aspect, the invention is directed to a method of treating pulmonary fibrosis by administering a TAFI inhibitor to a patient in need thereof.

In another aspect, the invention is directed to a method of treating pulmonary fibrosis using TAFI inhibitors of the following formula (I): 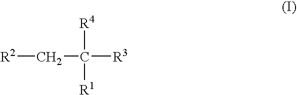
wherein:
R¹ is hydrogen, alkyl, alkenyl, aralkyl, or aralkenyl;
R² is —SH, —S—C(O)—R⁸, —P(O)(OR⁵)₂, —P(O)(OR⁵)R⁶, —P(O)(R⁵)—R⁷—N(R⁶)₂, —P(O)(OR⁵)—R⁷—C(O)—R⁸, —P(O)(OR⁵)—R⁷—N(R⁵)—C(O)OR⁸, —P(O)(OR⁵)—R⁷—N(R⁵)—C(O)—R⁷—N(R⁵)—C(O)OR⁸, —P(O)(OR⁵)—R⁷—N(R⁵)—S(O)₂—R⁹, or —P(O)(OR⁵)—R⁷—N(R⁵)—C(S)-N(R⁶)₂;
R³ is tetrazole, —C(O)OR⁶, —C(O)OR⁷—OC(O)R⁵, —S(O)OR⁵, —S(O)₂OR⁵, —P(O)(OR⁵)₂, —P(O)(OR⁵)R⁶, or —B(OR⁵)₂;
R⁴ is aryl optionally substituted by one or more substituents selected from the group consisting of alkyl, halo, haloalkyl, haloalkoxy, mercapto, alkylthio, phenyl, cycloalkyl, nitro, cyano, —OR⁶, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶);
provided that when R³ is —C(O)OH or when R⁴ is a substituted aryl or substituted N-heterocyclyl, R² can not be —P(O)(OR⁵)—R⁷—N(H)—C(O)OR⁸ or —P(Q)(OR⁵)—R⁷—N(H)—C(O)—R⁷—N(R⁵)—C(O)OR⁸;
   as a single stereoisomer, a mixture of stereoisomers, or as a racemic mixture of stereoisomers; or a pharmaceutically acceptable salt thereof.

In another aspect, the invention is directed to a method of treating pulmonary fibrosis using TAFI inhibitors of the following formula (II): 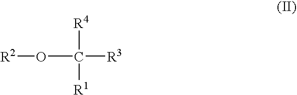
wherein:
R¹ is hydrogen, alkyl, alkenyl, aryl or aralkenyl;
R² is —P(O)(OR⁵)₂, —P(O)(OR⁵)R⁶, —P(O)(OR⁵)—R⁷—N(R⁶)₂, —P(O)(OR⁵)—R⁷—C(O)—R⁸, —P(Q)(OR⁵)—R⁷—N(R⁵)—C(O)OR⁸, —P(O)(OR⁵)—R⁷—N(R⁵)—C(O)—R⁷—N(R⁵)—C(O)OR⁸, —P(O)(OR⁵)—R⁷—N(R⁵)—S(O)₂—R⁹, or —P(O)(OR⁵)—R⁷—N(R⁵)—C(S)—N(R⁶)₂;
R³ is tetrazole, —C(O)O—R⁶, —C(O)O—R⁷—OC(O)R⁵, —S(O)OR⁵, —S(O)₂OR⁵, —P(O)(OR⁵)₂, —P(O)(OR⁵)R⁶, or —B(OR⁵)₂;
R⁴ is aryl optionally substituted by one or more substituents selected from the group consisting of alkyl, halo, haloalkyl, haloalkoxy, mercapto, alkylthio, phenyl, cycloalkyl, nitro, cyano, —OR⁶, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶);
provided that when R³ is —C(O)OH or when R⁴ is a substituted aryl or substituted N-heterocyclyl, R² can not be —P(O)(OR⁵)—R⁷—N(H)—C(O)OR⁸ or —P(O)(OR⁵)—R⁷—N(H)—C(O)—R⁷—N(R⁵)—C(O)OR⁸;
   as a single stereoisomer, a mixture of stereoisomers, or as a racemic mixture of stereoisomers; or a pharmaceutically acceptable salt thereof.

In another aspect, the invention is directed to a method of treating pulmonary fibrosis using TAFI inhibitors of formula (III): 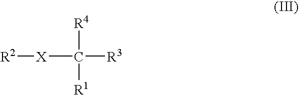
wherein:
X is —CH₂— or —O—;
R¹ is hydrogen, alkyl, alkenyl, aryl or aralkenyl;
R² is —P(O)(OR⁵)—R⁷—N(R⁵)—C(O)R⁶, —P(O)(OR⁵)—R⁷—N(R⁵)—C(O)OR⁸ or —P(O)(OR⁵)—R⁷—N(R⁵)—C(O)—R⁷—N(R⁵)—C(O)OR⁸,
R³ is —C(O)OH;
R⁴ is aryl optionally substituted by one or more substituents selected from the group consisting of alkyl, halo, haloalkyl, haloalkoxy, mercapto, alkylthio, phenyl, cycloalkyl, nitro, cyano, —OR⁶, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂); and
each R⁸ is independently alkyl, alkenyl, aryl, aralkyl or aralkenyl;
   as a single stereoisomer, a mixture of stereoisomers, or as a racemic mixture of stereoisomers; or a pharmaceutically acceptable salt thereof.

### DETAILED DESCRIPTION OF THE INVENTION

### A. DEFINITIONS

As used in the specification and appended claims, unless specified to the contrary, the following terms have the meaning indicated:

“Alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), and the like. Unless stated otherwise specifically in the specification, the alkyl radical may be optionally substituted by hydroxy, alkoxy, aryloxy, haloalkoxy, cyano, nitro, mercapto, alkylthio, cycloalkyl, —N(R⁶)₂, —C(O)OR⁶, —C(O)N(R⁶)₂ or —N(R⁶)—C(O)—R⁶ where each R⁶ is as defined in the Summary of the Invention. Unless stated otherwise specifically in the specification, it is understood that for radicals, as defined below, that contain a substituted alkyl group that the substitution can occur on any carbon-of the alkyl group.

“Alkoxy” refers to a radical of the formula —ORₐ where Rₐ is an alkyl radical as defined above, e.g., methoxy, ethoxy, n-propoxy, 1-methylethoxy (iso-propoxy), n-butoxy, n-pentoxy, 1,1-dimethylethoxy (t-butoxy), and the like. Unless stated otherwise specifically in the specification, it is understood that for radicals, as defined below, that contain a substituted alkoxy group that the substitution can occur on any carbon of the alkoxy group. The alkyl radical in the alkoxy radical may be optionally substituted as described above.

“Alkylthio” refers to a radical of the formula —SRₐ where Rₐ is an alkyl radical as defined above, e.g., methylthio, ethylthio, n-propylthio, 1-methylethylthio (iso-propylthio), n-butylthio, n-pentylthio, 1,1-dimethylethylthio (t-butylthio), and the like. Unless stated otherwise specifically in the specification, it is understood that for radicals, as defined below, that contain a substituted alkylthio group that the substitution can occur on any carbon of the alkylthio group. The alkyl radical in the alkylthio radical may be optionally substituted as described above.

“Alkenyl” refers to a straight or branched hydrocarbon chain radical consisting solely of carbon and hydrogen atoms, containing at least one double bond, having from two to eight carbon atoms, and which is attached to the rest of the molecule by a single bond or a double bond, e.g., ethenyl, prop-1-enyl, but-1-enyl, pent-1-enyl, penta-1,4-dienyl, and the like. Unless stated otherwise specifically in the specification, the alkenyl radical may be optionally substituted by hydroxy, alkoxy, haloalkoxy, cyano, nitro, mercapto, alkylthio, cycloalkyl, —N(R⁶)₂, —C(O)OR⁶, —C(O)N(R⁶)₂ or —N(R⁶)—C(O)—R⁶ where each R⁶ is as defined in the Summary of the Invention. Unless stated otherwise specifically in the specification, it is understood that for radicals, as defined below, that contain a substituted alkenyl group that the substitution can occur on any carbon of the alkenyl group.

“Alkynyl” refers to a straight or branched monovalent hydrocarbon chain radical consisting solely of carbon and hydrogen atoms, containing at least one triple bond, having from two to eight carbon atoms, and which is attached to the rest of the molecule by a single bond, e.g., ethynyl, prop-1-ynyl, but-1-ynyl, pent-1-ynyl, pent-3-ynyl, and the like. Unless stated otherwise specifically in the specification, the alkynyl radical may be optionally substituted by hydroxy, alkoxy, haloalkoxy, cyano, nitro, mercapto, alkylthio, cycloalkyl, —N(R⁶)₂, —C(O)OR⁶, —C(O)N(R⁶)₂ or —N(R⁶)—C(O)—R⁶ where each R⁶ is as defined in the Summary of the Invention. Unless stated otherwise specifically in the specification, it is understood that for radicals, as defined below, that contain a substituted alkynyl group that the substitution can occur on any carbon of the alkynyl group.

“Aryl” refers to a phenyl or naphthyl radical. Unless stated otherwise specifically in the specification, the term “aryl” or the prefix “ar-” (such as in “aralkyl”) is meant to include aryl radicals optionally substituted by one or more substituents selected from the group consisting of alkyl, halo, nitro, cyano, haloalkyl, haloalkoxy, mercapto, alkylthio, phenyl, cycloalkyl, —OR⁶ (including hydroxy and alkoxy), —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)OR⁶, —R⁷—C(O)OR⁶, —C(O)—N(R⁶)₂, —R⁷—C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂) where each R⁵, R⁶, and R⁷ are as defined above in the Summary of the Invention.

“Aralkyl” refers to a radical of the formula —RₐR_{b} where Rₐ is an alkyl radical as defined above and R_{b} is one or more aryl radicals as defined above, e.g., benzyl, diphenylmethyl and the like. The aryl radical(s) may be optionally substituted as described above.

“Aralkoxy” refers to a radical of the formula —OR_{d} where R_{d} is an aralkyl radical as defined above, e.g., benzyloxy, and the like. The aryl radical may be optionally substituted as described above.

“Aralkenyl” refers to a radical of the formula —R_{c}R_{b} where R_{c} is an alkenyl radical as defined above and R_{b} is one or more aryl radicals as defined above, e.g., 3-phenylprop-1-enyl, and the like. The aryl radical(s) and the alkenyl radical may be optionally substituted as described above.

“Alkylene chain” refers to a straight or branched divalent hydrocarbon chain consisting solely of carbon and hydrogen, containing no unsaturation and having from one to eight carbon atoms, e.g., methylene, ethylene, propylene, n-butylene, and the like. The alkylene chain may be optionally substituted by one or more substituents selected from the group consisting of aryl, halo, hydroxy, alkoxy, haloalkoxy, cyano, nitro, mercapto, alkylthio, cycloalkyl, —N(R⁶)₂, —C(O)OR⁶, —C(O)N(R⁶)₂ or —N(R⁶)—C(O)—R⁶ where each R⁶ is as described above in the Summary of the Invention. The alkylene chain may be attached to the rest of the molecule through any two carbons within the chain.

“Alkenylene chain” refers to a straight or branched divalent hydrocarbon chain consisting solely of carbon and hydrogen, containing at least one double bond and having from two to eight carbon atoms, e.g., ethenylene, prop-1-enylene, but-1-enylene, pent-1-enylene, hexa-1,4-dienylene, and the like. The alkenylene chain may be optionally substituted by one or more substituents selected from the group consisting of aryl, halo, hydroxy, alkoxy, haloalkoxy, cyano, nitro, mercapto, alkylthio, cycloalkyl, —N(R⁶)₂, —C(O)OR⁶, —C(O)N(R⁶)₂ or —N(R⁶)—C(O)—R⁶ where each R⁶ is as described above in the Summary of the Invention. The alkenylene chain may be attached to the rest of the molecule through any two carbons within the chain.

“Cycloalkyl” refers to a stable monovalent monocyclic or bicyclic hydrocarbon radical consisting solely of carbon and hydrogen atoms, having from three to ten carbon atoms, and which is saturated and attached to the rest of the molecule by a single bond, e.g., cyclopropyl, cyclobutyl, cyclopentyl, cyclohexyl, decalinyl and the like. Unless otherwise stated specifically in the specification, the term “cycloalkyl” is meant to include cycloalkyl radicals which are optionally substituted by one or more substituents independently selected from the group consisting of alkyl, aryl, aralkyl, halo, haloalkyl, hydroxy, alkoxy, haloalkoxy, cyano, nitro, mercapto, alkylthio, cycloalkyl, —N(R⁶)₂, —C(O)OR⁶, —C(O)N(R⁶)₂ or —N(R⁶)—C(O)—R⁶ where each R⁶ is as defined in the Summary of the Invention.

“Cycloalkylene” refers to a stable divalent monocyclic or bicyclic hydrocarbon consisting solely of carbon and hydrogen atoms, having from three to ten carbon atoms, and which is saturated and attached to the rest of the molecule by two single bonds, e.g., cyclopropylene, cyclobutylene, cyclopentylene, cyclohexylene, decalinylene and the like. Unless otherwise stated specifically in the specification, the term “cycloalkylene” is meant to include cycloalkylene moieties which are optionally substituted by one or more substituents independently selected from the group consisting of alkyl, aryl, aralkyl, halo, haloalkyl, hydroxy, alkoxy, haloalkoxy, cyano, nitro, mercapto, alkylthio, cycloalkyl, —N(R⁶)₂, —C(O)OR⁶, —C(O)N(R⁶)₂ or —N(R⁶)—C(O)—R⁶ where each R⁶ is as defined in the Summary of the Invention.

“N-heterocyclyl” refers to a stable 3- to 15-membered ring radical which consists of carbon atoms and from one to five heteroatoms selected from the group consisting of nitrogen, oxygen and sulfur wherein at least one of the heteroatoms is a nitrogen. For purposes of this invention, the N-heterocyclyl radical may be a monocyclic, bicyclic or tricyclic ring system, which may include fused or bridged ring systems; and the nitrogen, carbon or sulfur atoms in the N-heterocyclyl radical may be optionally oxidized; the nitrogen atom may be optionally quaternized; and the N-heterocyclyl radical may be partially or fully saturated or aromatic. The N-heterocyclyl radical may be attached to the main structure at any heteroatom or carbon atom which results in the creation of a stable compound. Examples of such N-heterocyclyl radicals include, but are not limited to, azepinyl, azetidinyl, benzimidazolyl, benzoxazolyl, carbazolyl, decahydroisoquinolyl, quinuclidinyl, imidazolyl, imidazolinyl, imidazolidinyl, isothiazolidinyl, indolyl, isoindolyl, indolinyl, isoindolinyl, indolizinyl, isoxazolyl, isoxazolidinyl, morpholinyl, benzothiadiazolyl, oxadiazolyl, octahydroindolyl, octahydroisoindolyl, 2-oxopiperazinyl, 2-oxopiperidinyl, 2-oxopyrrolidinyl, 2-oxoazepinyl, oxazolyl, oxazolidinyl, perhydroazepinyl, piperidinyl, piperazinyl, 4-piperidonyl, phenazinyl, phenothiazinyl, phenoxazinyl, phthalazinyl, pteridinyl, purinyl, pyrrolyl, pyrrolidinyl, pyrazolyl, pyrazolidinyl, pyridinyl, pyrazinyl, pyrimidinyl, pyridazinyl, quinazolinyl, quinoxalinyl, quinolinyl, quinuclidinyl, isoquinolinyl, thiazolyl, thiazolidinyl, thiadiazolyl, triazolyl, tetrazolyl, tetrahydroisoquinolyl, thiomorpholinyl, thiomorpholinyl sulfoxide, and thiomorpholinyl sulfone. The carbon atoms in the N-heterocyclyl radical may be optionally substituted by alkyl, halo, nitro, cyano, haloalkyl, haloalkoxy, mercapto, alkylthio, phenyl, cycloalkyl, —OR⁶, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)OR⁶, —R⁷—C(O)OR⁶, —C(O)—N(R⁶)₂, —R⁷—C(O)—N(R⁶)₂, —C(O)—R⁷—N(R⁵)₂, —N(R⁵)—C(NR⁵)—N(R⁵)₂, —N(R⁵)—C(O)—N(R⁶)₂ and —N(R⁵)—C(O)—R⁷—N(R⁶₂) where each R⁵, R⁶, R⁷ and R⁸ are as defined above in the Summary of the Invention. The nitrogen atoms in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂ where each R⁵, R⁶ and R⁷ are as defined above in the Summary of the Invention. Preferred N-heterocyclyl radicals are piperidinyl, tetrahydrosoquinolinyl, or benzothiadiazolyl.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like.

“Haloalkoxy” refers to a radical of the formula —OR_{c} where R_{c} is an haloalkyl radical as defined above, e.g., trifluoromethoxy, difluoromethoxy, trichloromethoxy, 2,2,2-trifluoroethoxy, 1-fluoromethyl-2-fluoroethoxy, 3-bromo-2-fluoropropoxy, 1-bromomethyl-2-bromoethoxy, and the like.

“Mammal” includes humans and domesticated animals, such as cats, dogs, swine, cattle, sheep, goats, horses, rabbits, and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

“Pulmonary Fibrosis” refers to all diseases of the lungs in which fibrosis plays a role. Pulmonary fibrosis includes lung fibrosis as well as interstitial lung disease.

“TAFI” refers to Thrombin Activatable Fibrinolysis Inhibitor, also known as plasma procarboxypeptidase B, which when activated gives rise to an active basic carboxypeptidase called activated TAFI or TAFIa. TAFIa is also known as carboxypeptidase U or carboxypeptidase R.

“Therapeutically effective amount” refers to that amount of a compound of the invention which, when administered to a human in need thereof, is sufficient to effect treatment, as defined below, for a disease-state characterized by thrombotic activity. The amount of a compound of the invention which constitutes a “therapeutically effective amount” will vary depending on the compound, the condition and its severity, and the age of the human to be treated, but can be determined routinely by one of ordinary skill in the art having regard to his own knowledge and to this disclosure.

“Treating” or “treatment” as used herein covers the treatment of a disease-state in a mammal, preferably a human, which disease-state is characterized by thrombotic activity, and includes:

(i) preventing the condition from occurring in a human, in particular, when such human is predisposed to the condition but has not yet been diagnosed as having it;

(ii) inhibiting the condition, i.e., arresting its development; or

(iii) relieving the condition, i.e., causing regression of the condition.

The compounds of the invention, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

The nomenclature used herein is a modified form of the I.U.P.A.C. nomenclature system wherein the compounds of the invention are named herein as derivatives of the acid moiety. For example, the following compound of formula (III) wherein R¹ is hydrogen, R² is —P(O)(OH)—R⁷—N(H)—C(O)OR⁸ (where R⁷ is hexyl and R⁸ is benzyl), R³ is —C(O)OH, and R⁴ is 3-guanidinophenyl, i.e., the compound of the following formula: 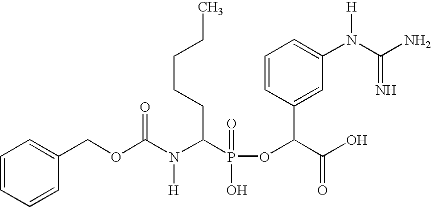
is named herein as 2-(3-guanidinophenyl)-2-((1-(benzyloxycarbonyl)aminohexyl)-(hydroxy)phosphinoyl)oxyethanoic acid. Unless otherwise indicated, compound names are intended to include any single stereoisomer, enantiomer, diastereomer, racemate or mixture of stereoisomers.

The use of parentheses in a formula herein is used to conserve space. Accordingly, the use of parenthesis in a formula indicates that the group enclosed within the parentheses is attached directly to the atom preceding the parenthesis. For example, the term —P(O)(OR⁵)—R⁷—N(R⁵)—C(O)—R⁷—N(R⁵)—C(O)OR⁸ can be drawn as follows:

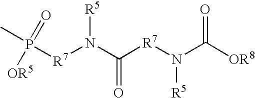

### B. UTILITY OF THE COMPOUNDS OF THE INVENTION

The compounds of the invention are inhibitors of TAFI and are therefore useful in treating pulmonary fibrosis, interstital lung diseases (ILD) and Acute Respiratory Distress Syndrome (ARDS).

The compounds of the invention may also be combined and/or coadministered with other therapeutic agents such as corticosteroids, interferon-gamma, pirfenidone, immunosupressive drugs, antithrombotics (including antiplatelet agents, anticoagulants and profbrinolytics), antihypertensives, agents to treat dyslipidaemia (e.g., statins such as LIPITOR™), anticoagulant activated protein C, Factor Xa inhibitors and antiarrhythmics (e.g., amiodarone and digoxin). Suitable antithrombotics include aspirin, clopidogrel, ticlopidine, warfarin, unfractionated heparin, hirudin, streptokinase, urokinase, recombinant tissue plasminogen activator (tPA), dipyridamole, REOPRO™, AGGRASTAT™, and INTEGRILIN™.

### C. ADMINISTRATION OF THE COMPOUNDS OF THE INVENTION

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the present invention may be in any form that allows for the composition to be administered to a patient. Typical routes of administration include, without limitation, oral, topical, transdermal, inhalation, parenteral, sublingual, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state characterized by thrombotic activity, i.e., by the formation of a thrombus, or by hypercoagulability, in accordance with the teachings of this invention.

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, e.g., inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, e.g., a gelatin capsule, it may contain, in addition to materials of the above type, a liquid carrier such as polyethylene glycol or a fatty oil.

The pharmaceutical composition may be in the form of a liquid, e.g., an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained. Typically, this amount is at least 0.01% of a compound of the invention in the composition. When intended for oral administration, this amount may be varied to be between 0.1 and about 70% of the weight of the composition. Preferred oral pharmaceutical compositions contain between about 4% and about 50% of the compound of the invention. Preferred pharmaceutical compositions and preparations according to the present invention are prepared so that a parenteral dosage unit contains between 0.01 to 1% by weight of the compound of the invention.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the invention from about 0.1 to about 10% w/v (weight per unit volume).

The pharmaceutical composition of the invention may be intended for rectal administration, in the form, e.g., of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term “aerosol” is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

Whether in solid, liquid or gaseous form, the pharmaceutical composition of the present invention may contain one or more known pharmacological agents used in the treatment of disease-states characterized by thrombotic activity.

The pharmaceutical compositions of the invention may be prepared by methodology well known in the pharmaceutical art. For example, a pharmaceutical composition intended to be administered by injection can be prepared by combining a compound of the invention with water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disease-state; and the host undergoing therapy. Generally, a therapeutically effective daily dose is from about 0.14 mg to about 14.3 mg/kg of body weight per day of a compound of the invention, or a pharmaceutically acceptable salt thereof; preferably, from about 0.7 mg to about 10 mg/kg of body weight per day; and most preferably, from about 1.4 mg to about 7.2 mg/kg of body weight per day. For example, for administration to a 70 kg person, the dosage range would be from about 10 mg to about 1.0 gram per day of a compound of the invention, or a pharmaceutically acceptable salt thereof, preferably from about 50 mg to about 700 mg per day, and most preferably from about 100 mg to about 500 mg per day.

### D. PREFERRED EMBODIMENTS

Of the compounds of the invention as set forth above in the Summary of the Invention, several groups of compounds are particularly preferred.

Of the compounds of formula (I) as set forth above in the Summary of the Invention, a preferred group is that group of compounds of formula (I) wherein:
R¹ is hydrogen;
R² is —SH or —S—C(O)—R⁸;
R³ is tetrazole, —C(O)OR⁶ or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or —C(O)N(R⁶)₂; and
each R⁸ is independently alkyl, alkenyl, aryl, aralkyl or aralkenyl.

Of this preferred group of compounds, a preferred subgroup is that subgroup of compounds of formula (I) wherein:
R¹ is hydrogen;
R² is —SH or —S—C(O)—R⁸;
R³ is —C(O)OR⁶;
R⁴ is aryl optionally substituted by one or more substituents selected from the group consisting of halo, nitro, —N(R⁶)₂, —R⁷—N(R⁶)₂ and —N(R⁵)—C(NR⁵)—N(R⁵)₂;
each R⁵ is independently hydrogen, alkyl or aralkyl;
each R⁶ is independently hydrogen, alkyl, aryl or aralkyl;
R⁷ is a straight or branched alkylene chain; and
R⁸ is independently alkyl, alkenyl, aryl, aralkyl or aralkenyl.

Of this preferred subgroup of compounds of formula (I), preferred compounds are selected from the group consisting of the following:
2-(4-guanidinophenyl)-3-mercaptopropanoic acid;
2-(3-guanidinophenyl)-3-mercaptopropanoic acid;
2-(3-aminophenyl)-3-mercaptopropanoic acid; and
2-(2-chloro-5-guanidinophenyl)-3-mercaptopropanoic acid.

Of the preferred group of compounds of formula (I) as set forth above, another preferred subgroup is that subgroup of compounds of formula (I) wherein:
R¹ is hydrogen;
R² is —SH, or —S—C(O)—R⁸;
R³ is —C(O)OR⁶;
R⁴ is 3(4)-piperidinyl wherein the nitrogen atom in the piperidinyl radical is optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, aryl or aralkyl;
R⁷ is a straight or branched alkylene chain optionally substituted by hydroxy, mercapto, alkylthio, aryl, cycloalkyl, —N(R⁶)₂, —C(O)OR⁶, or —C(O)N(R⁶)₂; and
R⁸ is alkyl, alkenyl, aryl, aralkyl or aralkenyl.

Of this preferred subgroup of compounds of formula (I), preferred compounds are selected from the group consisting of the following:
2-(piperidin-4-yl)-3-mercaptopropanoic acid;
2-(1-amidinopiperidin-4-yl)-3-mercaptopropanoic acid;
2-(1-(1-iminoethyl)piperidin-4-yl)-3-mercaptopropanoic acid;
2-(1-(aminomethylcarbonyl)piperidin-4-yl)-3-mercaptopropanoic acid;
2-(piperidin-3-yl)-3-mercaptopropanoic acid; and
2-(1-amidinopiperidin-3-yl)-3-mercaptopropanoic acid.

Of the compounds of formula (I) as set forth above in the Summary of the Invention, another preferred group is that group of compounds of formula (I) wherein:
R¹ is hydrogen;
R² is —P(O)(OR⁵)₂, —P(O)(OR⁵)R⁶ or —P(O)(OR⁵)—R⁷—C(O)—R⁸;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or —C(O)N(R⁶)₂; and
each R⁸ is independently alkyl, alkenyl, aryl, aralkyl or aralkenyl.

Of this preferred group of compounds, a preferred subgroup is that subgroup of compounds of formula (I) wherein:
R¹ is hydrogen;
R² is —P(O)(OR⁵)₂, —P(O)(OR⁵)R⁶ or —P(O)(OR⁵)—R⁷—C(O)—R⁸;
R³ is —C(O)OR⁶;
R⁴ is aryl optionally substituted by one or more substituents selected from the group consisting of halo, nitro, —N(R⁶)₂, —R⁷—N(R⁶)₂ and —N(R⁵)-C(NR⁵)—N(R⁵)₂;
each R⁵ is independently hydrogen, alkyl or aralkyl;
each R⁶ is independently hydrogen, alkyl, aryl or aralkyl;
each R⁷ is independently a straight or branched alkylene chain optionally substituted by aryl, —N(R⁶)₂ or —C(O)OR⁶; and
R⁸ is alkyl, alkenyl, aryl, aralkyl or aralkenyl.

Of this preferred subgroup of compounds of formula (I), preferred compounds are selected from the group consisting of the following:
2-(3-guanidinophenyl)-3-phosphonopropanoic acid;
2-(3-aminophenyl)-3-((phenyl)(hydroxy)phosphinoyl)propanoic acid;
2-(3-aminophenyl)-3-((4-phenylbutyl)(hydroxy)phosphinoyl)propanoic acid;
2-(3-aminophenyl)-3-((pentyl)(hydroxy)phosphinoyl)propanoic acid;
2-(3-guanidinophenyl)-3-((phenyl)(hydroxy)phosphinoyl)propanoic acid;
2-(3-guanidinophenyl)-3-((4-phenylbutyl)(hydroxy)phosphinoyl)propanoic acid;
2-(3-guanidinophenyl)-3-((pentyl)(hydroxy)phosphinoyl)propanoic acid;
2-(3-guanidinophenyl)-3-((4-methylpentyl)(hydroxy)phosphinoyl)propanoic acid;
2-(3-guanidinophenyl)-3-((3-phenylpropyl)(hydroxy)phosphinoyl)propanoic acid;
2-(3-guanidinophenyl)-3-((3-phenylprop-2-enyl)(hydroxy)phosphinoyl)propanoic acid;
2-(3-guanidinophenyl)-3-((phenylmethyl)(hydroxy)phosphinoyl)propanoic acid;
2-(3-guanidinophenyl)-3-((pentyl)(hydroxy)phosphinoyl)propanoic acid methyl ester;
2-(3-guanidinophenyl)-3-((ethyl)(hydroxy)phosphinoyl)propanoic acid;
2-(3-guanidinophenyl)-3-((2-phenylethyl)(hydroxy)phosphinoyl)propanoic acid; and
2-(3-guanidinophenyl)-3-((2-(methylcarbonyl)ethyl)(hydroxy)phosphinoyl)propanoic acid.

Of the compounds of formula (I) as set forth above in the Summary of the Invention, another preferred group is that group of compounds of formula (I) wherein:
R¹ is hydrogen;
R² is —P(O)(OR⁵)—R⁷—N(R⁵)—C(O)OR⁸;
R³ is —C(O)OR⁶ (where R⁶ is alkyl, aryl or aralkyl);
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶—C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂) where each R⁶ is independently hydrogen, alkyl, aryl or aralkyl;
each R⁵ is independently hydrogen, alkyl or aralkyl;
each R⁷ is a straight or branched alkylene chain optionally substituted by aryl, —N(R⁶)₂ or —C(O)OR⁶; and
each R⁸ is independently alkyl, alkenyl, aryl, aralkyl or aralkenyl.

Of this group of compounds of formula (I), preferred compounds are selected from the group consisting of the following:
2-(3-(t-butoxycarbonylamino)methylphenyl)-3-((1-(benzyloxycarbonyl)amino-2-methylpropyl)(hydroxy)phosphinoyl)propanoic acid t-butyl ester; and
2-(3-(t-butoxycarbonylamino)methylphenyl)-3-((1-(benzyloxycarbonyl)amino-2-methylpropyl)(ethoxy)phosphinoyl)propanoic acid t-butyl ester.

Of the compounds of formula (I) as set forth above in the Summary of the Invention, another preferred group is that group of compounds of formula (I) wherein:
R¹ is hydrogen;
R² is —P(O)(OR⁵)—R⁷—N(R⁶)₂ or —P(O)(OR⁵)—R⁷—N(R⁵)—C(S)—N(R⁶)₂;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
each R⁵ is independently hydrogen, alkyl or aralkyl;
each R⁶ is independently hydrogen, alkyl, alkenyl, alkynyl, aryl, aralkyl or aralkenyl;
each R⁷ is a straight or branched alkylene chain optionally substituted by hydroxy, mercapto, alkylthio, aryl, cycloalkyl, —N(R⁶)₂, —C(O)OR , or —C(O)N(R⁶)₂; and
each R⁸ is independently alkyl, alkenyl, aryl, aralkyl or aralkenyl.

Of this group of compounds of formula (I), preferred compounds are selected from the group consisting of the following:
2-(3-(t-butoxycarbonylamino)methylphenyl)-3-((1-amino-2-methylpropyl)(hydroxy)-phosphinoyl)propanoic acid t-butyl ester; and
2-(3-(t-butoxycarbonylamino)methylphenyl)-3-((1-amino-2-methylpropyl)(ethoxy)-phosphinoyl)propanoic acid t-butyl ester.

Of the compounds of formula (I) as set forth above in the Summary of the Invention, another preferred group is that group of compounds of formula (I) wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR , —C(O)N(R⁶)₂ or —N(R⁶)C(O)R⁶).

Of this group of compounds, a preferred subgroup is that subgroup of compounds of formula (I) wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, and —N(R⁵)—C(NR⁵)—N(R⁵)₂;
each R⁵ is independently hydrogen, alkyl or aralkyl;
each R⁵ is independently hydrogen, alkyl, alkenyl, alkynyl, aryl, aralkyl or aralkenyl;
each R⁷ is independently a straight or branched alkylene chain (optionally substituted by hydroxy, mercapto, alkylthio, aryl, cycloalkyl, —N(R⁶)₂, —C(O)OR⁶, or —C(O)N(R⁶)₂),
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

Of this preferred subgroup of compounds, a preferred class is that class of compounds of formula (I) wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or —C(O)N(R⁶)₂,
each R⁸ is independently alkyl, alkenyl, aryl, aralkyl or aralkenyl; and
R⁹ is alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

Of this preferred class of compounds of formula (I), preferred compounds are selected from the group consisting of the following:
2-(3-(amino)methylphenyl)-3-((1-(3-phenylpropylsulfonyl)amino-2-methylpropyl)-(hydroxy)phosphinoyl)propanoic acid, methyl ester;
2-(3-(t-butoxycarbonylamino)methylphenyl)-3-((1-(3-phenylpropylsulfonyl)amino-2-methylpropyl)(hydroxy)phosphinoyl)propanoic acid;
2-(3-(t-butoxycarbonylamino)methylphenyl)-3-((1-(3-phenylpropylsulfonyl)amino-2-methylpropyl)(hydroxy)phosphinoyl)propanoic acid, methyl ester;
2-(3-(amino)methylphenyl)-3-((1-(3-phenylpropylsulfonyl)amino-2-methylpropyl)(hydroxy )-phosphinoyl)propanoic acid;
(2R)-2-(3-(amino)methylphenyl)-3-(((1R)-1-(3-phenylpropylsulfonyl)amino-2-methylpropyl)(hydroxy)phosphinoyl)propanoic acid;
(2S)-2-(3-(amino)methylphenyl)-3-(((1R)-1-(3-phenylpropylsulfonyl)amino-2-methylpropyl)(hydroxy)phosphinoyl)propanoic acid;
(2R/S)-2-(3-(amino)methylphenyl)-3-(((1S)-1-(3-phenylpropylsulfonyl)amino-2-methylpropyl)(hydroxy)phosphinoyl)propanoic acid;
(2R/S)-2-(3-(amino)methylphenyl)-3-(((1R)-1-(3-phenylpropylsulfonyl)amino-2-methylpropyl)(hydroxy)phosphinoyl)propanoic acid;
(2R)-2-(3-(amino)methylphenyl)-3-(((1S)-1-(3-phenylpropylsulfonyl)amino-2-methylpropyl)(hydroxy)phosphinoyl)propanoic acid;
(2S)-2-(3-(amino)methylphenyl)-3-(((1S)-1-(3-phenylpropylsulfonyl)amino-2-methylpropyl)(hydroxy)phosphinoyl)propanoic acid;
2-(3-(t-butoxycarbonylamino)methylphenyl)-3-((1-(3-phenylpropylsulfonyl)amino-2-methylpropyl)(ethoxy)phosphinoyl)propanoic acid, t-butyl ester;
2-(3-(amino)methylphenyl)-3-((1-(2-phenylethylsulfonyl)amino-2-methylpropyl)-(hydroxy)phosphinoyl)propanoic acid;
2-(3-(amino)methylphenyl)-3-((1-(benzylsulfonyl)amino-2-methylpropyl)-(hydroxy)phosphinoyl)propanoic acid;
2-(3-(amino)methylphenyl)-3-((1-(2-(naphth-1-yl)ethylsulfonyl)amino-2-methylpropyl)(hydroxy)phosphinoyl)propanoic acid;
2-(3-(amino)methylphenyl)-3-((1-(3-(4-methoxyphenyl)propylsulfonyl)amino-2-methylpropyl)(hydroxy)phosphinoyl)propanoic acid;
2-(3-(amino)methylphenyl)-3-((1-(2-(4-methoxyphenyl)ethylsulfonyl)amino-2-methylpropyl)(hydroxy)phosphinoyl)propanoic acid;
2-(3-(amino)methylphenyl)-3-((1-(methylsulfonyl)amino-2-methylpropyl)(hydroxy)phosphinoyl)propanoic acid;
2-(3-(amino)methylphenyl)-3-((1-(2-benzyloxyethylsulfonyl)amino-2-methylpropyl)-(hydroxy)phosphinoyl)propanoic acid;
2-(3-(amino)methylphenyl)-3-((1-(2-hydroxyethylsulfonyl)amino-2-methylpropyl)-(hydroxy)phosphinoyl)propanoic acid;
2-(3-aminophenyl)-3-((1-(3-phenylpropylsulfonyl)amino-2-methylpropyl)-(hydroxy)phosphinoyl)propanoic acid;
2-(3-guanidinophenyl)-3-((1-(3-phenylpropylsulfonyl)amino-2-methylpropyl)(hydroxy)-phosphinoyl)propanoic acid;
2-(3-(amino)methylphenyl)-3-((1-(4-phenylbutylsulfonyl)amino-2-methylpropyl)-(hydroxy)phosphinoyl)propanoic acid, and
2-(3-(amino)methylphenyl)-3-((1-(2-phenylethenylsulfonyl)amino-2-methylpropyl)-(hydroxy)phosphinoyl)propanoic acid.

Of the preferred subgroup of compounds as set forth above, another preferred class is that class of compounds of formula (I) wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or —C(O)N(R⁶)₂,
each R⁸ is independently alkyl, alkenyl, aryl, aralkyl or aralkenyl; and
R⁹ is aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

Of this class of compounds of formula (I), preferred compounds are selected from the group consisting of the following:
2-(3-(amino)methylphenyl)-3-((1-(naphth-1-ylsulfonyl)amino-2-methylpropyl)(hydroxy)-phosphinoyl)propanoic acid;
2-(3-(amino)methylphenyl)-3-((1-(3-trifluoromethylphenylsulfonyl)amino-2-methyl-propyl)(hydroxy)phosphinoyl)propanoic acid;
2-(3-(amino)methylphenyl)-3-((1-(4-pentylphenylsulfonyl)amino-2-methylpropyl)-(hydroxy)phosphinoyl)propanoic acid;
2-(3-(amino)methylphenyl)-3-((1-(4-acetamidophenylsulfonyl)amino-2-methylpropyl)-(hydroxy)phosphinoyl)propanoic acid;
2-(3-(amino)methylphenyl)-3-((1-(4-phenylphenylsulfonyl)amino-2-methylpropyl)-(hydroxy)phosphinoyl)propanoic acid; and
2-(3-(amino)methylphenyl)-3-((1-(phenylsulfonyl)amino-2-methylpropyl)(hydroxy)-phosphinoyl)propanoic acid.

Of the preferred subgroup of compounds as set forth above, another preferred class is that class of compounds of formula (I) wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, —N(R⁶)—C(O)—R⁶, —R⁷—N(R⁶)—C(O)—R⁶, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain (optionally substituted by hydroxy, mercapto, alkylthio, aryl, cycloalkyl, —N(R⁶)₂, —C(O)OR⁶, or —C(O)N(R⁶)₂),
each R⁸ is independently alkyl, alkenyl, aryl, aralkyl or aralkenyl; and
R⁹ is —R⁷—N(R⁶)—C(O)OR⁸.

Of this class of compounds of formula (I), a preferred compound is 2-(3-(amino)methylphenyl)-3-((1-(3-phenyl-2-(benzyloxycarbonyl)aminopropyl-sulfonyl)amino-2-methylpropyl)(hydroxy)phosphinoyl)propanoic acid.

Of the preferred subgroup of compounds as set forth above, another preferred class is that class of compounds of formula (I) wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, —N(R⁶)—C(O)—R⁶, —R⁷—N(R⁶)—C(O)—R⁶, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain (optionally substituted by hydroxy, mercapto, alkylthio, aryl, cycloalkyl, —N(R⁶)₂, —C(O)OR⁶, or —C(O)N(R⁶)₂),
each R⁸ is independently alkyl, alkenyl, aryl, aralkyl or aralkenyl; and
R⁹ is N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

Of this class of compounds of formula (I), preferred compounds are selected from the group consisting of the following:
2-(3-(amino)methylphenyl)-3-((1-(thien-2-ylsulfonyl)amino-2-methylpropyl)-(hydroxy)phosphinoyl)propanoic acid; and
2-(3-(amino)methylphenyl)-3-((1-(benzothiadiazolylsulfonyl)amino-2-methylpropyl)-(hydroxy)phosphinoyl)propanoic acid.

Of the compounds of formula (I) as set forth above in the Summary of the Invention, another preferred group is that group of compounds of formula (I) wherein:
R¹ is hydrogen;
R² is —P(O)(OR⁵)—R⁷—N(R⁵)—C(O)OR⁸;
R is —C(O)OR⁶;
R⁴ is unsubstituted phenyl or unsubstituted N-heterocyclyl;
each R⁵ is independently hydrogen, alkyl or aralkyl;
each R⁶ is independently hydrogen, alkyl, alkenyl, alkynyl, aryl, aralkyl or aralkenyl;
each R⁷ is a straight or branched alkylene chain optionally substituted by aryl, —N(R⁶)₂ or —C(O)OR⁶; and
R⁸ is alkyl, alkenyl, aryl, aralkyl or aralkenyl.

Of this group of compounds of formula (I), preferred compounds are selected from the group consisting of the following:
2-phenyl-3-((1-(benzyloxycarbonyl)amino-2-methylpropyl)(hydroxy)phosphinoyl)-propanoic acid;
2-tetrahydroisoquinolinyl-3-((1-(benzyloxycarbonyl)amino-2-methylpropyl)-(hydroxy)phosphinoyl)propanoic acid.

Of the compounds of formula (II) as set forth above in the Summary of the Invention, a preferred group is that group of compounds of formula (II) wherein:
R¹ is hydrogen;
R² is —P(O)COR⁵)R⁶, —P(O)(OR⁵)—R⁷—N(R⁶)₂, or —P(O)(OR⁵)—R⁷—N(R⁵)—C(S)—N(R⁶)₂;
R³ is tetrazole, —C(O)OR⁶ or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
each R⁵ is independently hydrogen, alkyl or aralkyl;
each R⁶ is independently hydrogen, alkyl, alkenyl, alkynyl, aryl, aralkyl or aralkenyl;
each R⁷ is a straight or branched alkylene chain (optionally substituted by hydroxy, mercapto, alkylthio, aryl, cycloalkyl, —N(R⁶)₂, —C(O)OR⁶, or —C(O)N(R⁶)₂); and
each R⁸ is independently alkyl, alkenyl, aryl, aralkyl or aralkenyl.

Of this group of compounds of formula (II), preferred compounds are selected from the group consisting of the following:
2-(3-guanidinophenyl)-2-((1-(2-phenylethyl)amino-2-methylpropyl)-(hydroxy)phosphinoyloxy)ethanoic acid;
2-(3-aminophenyl)-2-((phenyl)(hydroxy)phosphinoyloxy)ethanoic acid;
2-(3-guanidinophenyl)-2-((1-amino-2-methylpropyl)(hydroxy)phosphinoyloxy)ethanoic acid; and
2-(3-guanidinophenyl)-2-((1-(benzylaminothiocarbonyl)amino-2-methylpropyl)-(hydroxy)phosphinoyloxy)ethanoic acid.

Of the compounds of formula (II) as set forth above in the Summary of the Invention, another preferred group is that group of compounds of formula (II) wherein:
R¹ is hydrogen;
R² is —P(O)(OR⁵)—R⁷—N(R⁵)—S(O)₂—R⁹;
R³ is tetrazole, —C(O)OR⁶ or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
each R⁵ is independently hydrogen, alkyl or aralkyl;
each R⁶ is independently hydrogen, alkyl, alkenyl, alkynyl, aryl, aralkyl or aralkenyl;
each R⁷ is a straight or branched alkylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

Of this group of compounds of formula (II), preferred compounds are selected from the group consisting of the following:
2-(3-guanidinophenyl)-2-((1-(benzylsulfonyl)amino-2-methylpropyl)(hydroxy)-phosphinoyloxy)ethanoic acid; and
2-(3-guanidinophenyl)-2-((1-(2-phenylethenylsulfonyl)amino-2-methylpropyl)-(hydroxy)phosphinoyloxy)ethanoic acid.

Of the compounds of formula (II) as set forth above in the Summary of the Invention, another preferred group is that group of compounds of formula (II) wherein:
R¹ is hydrogen;
R² is —P(O)(OR⁵)₂, —P(O)(OR⁵)R⁶, —P(O)(OR⁵)—R⁷—N(R⁶)₂—P(O)(OR⁵)—R⁷—C(O)—R⁸, —P(O)(OR⁵)—R⁷—N(R⁵)—C(O)OR⁸, —P(O)(OR⁵)—R⁷—N(R⁵)—C(O)—R⁷—N(R⁵)—C(O)OR⁸, —P(O)(OR⁵)—R⁷—N(R⁵)—S(O)₂—R⁹, or —P(O)(OR⁵)—R⁷—N(R⁵)—C(S)—N(R⁶)₂;
R³ is tetrazole;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

Of this group of compounds of formula (II), a preferred compound is 2-methyl-1-[1-(3-guanidinophenyl)-1-tetrazolylmethoxy](hydroxy)phosphinoyl-propylcarbamic acid, benzyl ester.

Of the compounds of formula (III) as set forth above in the Summary of the Invention, a preferred group is that group of compounds of formula (III) wherein:
X is —O—;
R² is —P(O)(OR⁵)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂).

Of this group of compounds of formula (III), preferred compounds are selected from the group consisting of the following:
2-(3-guanidinophenyl)-2-((1-(benzyloxycarbonyl)aminoethyl)(hydroxy)-phosphinoyloxy)ethanoic acid;
2-(3-guanidinophenyl)-2-(((benzyloxycarbonyl)aminomethyl)(hydroxy)-phosphinoyloxy)ethanoic acid;
2-(3-guanidinophenyl)-2-((1-(benzyloxycarbonyl)amino-2-methylpropyl)(hydroxy)-phosphinoyloxy)ethanoic acid;
2-(3-guanidinophenyl)-2-((1-(benzyloxycarbonyl)aminohexyl)(hydroxy)-phosphinoyloxy)ethanoic acid;
2-(3-aminophenyl)-2-((1-(benzyloxycarbonyl)amino-2-methylpropyl)-(hydroxy)phosphinoyloxy)ethanoic acid,
2-(3-guanidinophenyl)-2-((1-(benzyloxycarbonyl)amino-3-methylbutyl)-(hydroxy)phosphinoyloxy)ethanoic acid;
2-(2-chloro-3-guanidinophenyl)-2-((1-(benzyloxycarbonyl)amino-2-methylpropyl)-(hydroxy)phosphinoyloxy)ethanoic acid;
2-(3-guanidinophenyl)-2-((1-(benzyloxycarbonyl)amino-1-phenylmethyl)-(hydroxy)phosphinoyloxy)ethanoic acid;
2-(2-fluoro-3-guanidinophenyl)-2-((1-(benzyloxycarbonyl)amino-2-methylpropyl)-(hydroxy)phosphinoyloxy)ethanoic acid;
2-(3-guanidinophenyl)-2-((1-(benzyloxycarbonyl)amino-1-cyclohexylmethyl)-(hydroxy)phosphinoyloxy)ethanoic acid;
2-(2-methyl-3-guanidinophenyl)-2-((1-(benzyloxycarbonyl)amino-2-methylpropyl)-(hydroxy)phosphinoyloxy)ethanoic acid;
2-(3-(amino)methylphenyl)-2-((1-(benzyloxycarbonyl)amino-2-methylpropyl)-(hydroxy)phosphinoyloxy)ethanoic acid;
2-(3-(guanidinomethyl)phenyl)-2-((1-(benzyloxycarbonyl)amino-2-methylpropyl)-(hydroxy)phosphinoyloxy)ethanoic acid;
2-(3-(1-iminoethylaminophenyl))-2-((1-(benzyloxycarbonyl)amino-2-methylpropyl)-(hydroxy)phosphinoyloxy)ethanoic acid;
2-(3-(t-butoxycarbonylamino)methylphenyl)-2-((1-(benzyloxycarbonyl)amino-2-methylpropyl)(hydroxy)phosphinoyloxy)ethanoic acid;
2-(3-(ethoxycarbonylamino)methylphenyl)-2-((1-(benzyloxycarbonyl)amino-2-methylpropyl)(hydroxy)phosphinoyloxy)ethanoic acid;
2-(3-(isopropoxycarbonylamino)methylphenyl)-2-((1-(benzyloxycarbonyl)amino-2-methylpropyl)(hydroxy)phosphinoyloxy)ethanoic acid;
2-(3-(2,2-dimethylpropylcarbonylamino)methylphenyl)-2-((1-(benzyloxycarbonyl)-amino-2-methylpropyl)(hydroxy)phosphinoyloxy)ethanoic acid;
2-(3-guanidinophenyl)-2-((1-(2-phenylethylcarbonyl)amino-2-methylpropyl)-(hydroxy)phosphinoyloxy)ethanoic acid; and
2-(3-guanidinophenyl)-2-((1-(2-phenylethenylcarbonyl)amino-2-methylpropyl)-(hydroxy)phosphinoyloxy)ethanoic acid.

Of the compounds of formula (III) as set forth above in the Summary of the Invention, another preferred group is that group of compounds of formula (III) wherein:
X is —O—;
R² is —P(O)(OR⁵)—R⁷—N(R⁵)—C(O)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR—, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂).

Of this group of compounds of formula (III), preferred compounds are selected from the group consisting of the following:
2-(3-guanidinophenyl)-2-[(1-(1-benzyloxycarbonylamino-2-(4-hydroxyphenyl)-ethylcarbonyl)amino-2-methylpropyl)(hydroxy)phosphinoyloxy]ethanoic acid;
2-(3-guanidinophenyl)-2-[(1-(1-benzyloxycarbonylamino-2-phenylethylcarbonyl)amino-2-methylpropyl)(hydroxy)phosphinoyloxy]ethanoic acid;
2-(2-fluoro-3-guanidinophenyl)-2-[(1-(1-benzyloxycarbonylamino-2-phenyl-ethylcarbonyl)amino-2-methylpropyl)(hydroxy)phosphinoyloxy]ethanoic acid;
2-(3-guanidinophenyl)-2-[(1-(1-phenylcarbonylamino-2-phenylethylcarbonyl)amino-2-methylpropyl)(hydroxy)phosphinoyloxy]ethanoic acid;
2-(3-guanidinophenyl)-2-[(1-(1-ethoxycarbonylamino-2-phenylethylcarbonyl)amino-2-methylpropyl)(hydroxy)phosphinoyloxy]ethanoic acid;
2-(3-guanidinophenyl)-2-[(1-(1-benzyloxycarbonylamino-3-phenylpropyl-carbonyl)amino-2-methylpropyl)(hydroxy)phosphinoyloxy]ethanoic acid; and
2-(3-(amino)methylphenyl)-2-[(1-(1-benzyloxycarbonylamino-3-phenylpropyl-carbonyl)amino-2-methylpropyl)(hydroxy)phosphinoyloxy]ethanoic acid.

Of the compounds of formula (III) as set forth above in the Summary of the Invention, another preferred group is that group of compounds of formula (III) wherein:
X is —CH₂—;
R² is —P(O)(OR⁵)—R⁷—N(R⁵)—C(O)R⁶ or —P(O)(OR⁵)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂).

Of this group of compounds of formula (III), preferred compounds are selected from the group consisting of the following:
2-(3-(amino)methylphenyl)-3-((1-(methylcarbonyl)amino-2-methylpropyl)(hydroxy)-phosphinoyl)propanoic acid;
2-(3-(hydrazinocarbonyl)phenyl)-3-((1-(benzyloxycarbonyl)amino-2-methylpropyl)-(hydroxy)phosphinoyl)propanoic acid;
2-(3-guanidinophenyl)-3-((1-(benzyloxycarbonyl)amino-3-methylbutyl)-(hydroxy)phosphinoyl)propanoic acid;

2-(3-guanidinophenyl)-3-(((benzyloxycarbonyl)aminomethyl)(hydroxy)-phosphinoyl)propanoic acid;
2-(3-guanidinophenyl)-3-((1-(benzyloxycarbonyl)aminoethyl)(hydroxy)-phosphinoyl)propanoic acid;
2-(3-guanidinophenyl)-3-((1-(benzyloxycarbonyl)amino-2-methylpropyl)(hydroxy)-phosphinoyl)propanoic acid;
2-(2-chloro-5-guanidinophenyl)-3-((1-(benzyloxycarbonyl)amino-2-methylpropyl)(hydroxy)phosphinoyl)propanoic acid;
2-(3-(amino)methylphenyl)-3-((1-(benzyloxycarbonyl)amino-2-methylpropyl)(hydroxy)-phosphinoyl)propanoic acid; and
2-(3-(amino)methylphenyl)-3-((1-(2-phenylethylcarbonyl)amino-2-methylpropyl)(hydroxy)phosphinoyl)propanoic acid.

Of the compounds of formula (III) as set forth above in the Summary of the Invention, another preferred group is that group of compounds of formula (III) wherein:
X is —CH₂—;
R² is —P(O)(OR⁵)—R⁷—N(R⁵)—C(O)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂).

Of this group of compounds of formula (III), preferred compounds are selected from the group consisting of the following:
2-(3-guanidinophenyl)-3-(((1-benzyloxycarbonylamino-2-phenylethyl)-carbonylaminomethyl)(hydroxy)phosphinoyl)propanoic acid; and
2-(3-guanidinophenyl)-3-(((1-benzyloxycarbonylamino-2-phenylethyl)-carbonylaminomethyl)(hydroxy)phosphinoyl)propanoic acid.

The protective role of the plasminogen-plasmin activation system against lung fibrosis has been well-documented (Swaisgood et al., supra). The effector enzyme of the fibrinolysis system is plasmin, which results from the activation of plasminogen by tissue-type (t-PA) or urokinase-type (uPA) plasminogen activators (Saksela et al., supra). Plasmin is the key enzyme for preventing the development of pulmonary fibrosis because it lyses intraalveolar deposits of fibrin, degrades matrix components and activates the precursors of several metalloproteinases. Previous studies have clearly demonstrated that low intraalveolar plasmin activity is associated with the occurrence of pulmonary fibrosis (Hattori et al., supra; Loskutoff et al., supra; Chapman, H A, “Disorders of lung matrix remodeling”, J. Clin Invest. 113:148-57 (2004); Idell S., et al., “Abnormalities of pathways of fibrin turnover in lung lavage of rats with oleic acid and bleomycin-induced lung injury support alveolar fibrin deposition”, Am. J Pathol. 135:387-99 (1989)). Inhibition of the fibrinolytic activity may occur due to suppression of plasminogen activators by the specific plasminogen activator inhibitors, PAI-1 and PAI-2, direct inhibition of plasmin by its specific inhibitor alpha2-antiplasmin or due to decreased generation of the ternary complex formed by binding of plasminogen and t-PA PA on fibrin surface. This latter mechanism depends on TAFI activity (Bajzar, supra). Upon activation by thrombin, the thrombin/thrombomodulin complex or plasmin, TAFI decreases the binding of plasminogen to fibrin surface by removing the carboxy-terminal arginine and lysine residues from partially degraded fibrin, thereby reducing the formation of the t-PA, plasminogen and fibrin tertiary complex and subsequently the production of plasmin (Bajzar, supra). The role of lung fibrosis-associated hypofibrinolysis induced by plasminogen activator inhibitors and alpha2-antiplasmin has been well demonstrated but that mediated by TAFI remains unclear. In our study, the results of which are set forth below under the section headed EXAMPLES, we hypothesized that TAFI-induced low plasmin generation is also implicated in the mechanism of lung fibrosis. To demonstrate this hypothesis, we induced lung fibrosis in TAFI (+/+), TAFI (+/−) and TAFI (−/−) mice by chronic subcutaneous administration of bleomycin. This model was chosen because low fibrinolytic activity has been also implicated in the pathogenesis of lung fibrosis induced by bleomycin in the mouse.

Inflammation is the initial response to lung injury. The inflammatory response is characterized by the recruitment of macrophages, neutrophils, lymphocytes and eosinophils within the alveolar and interstitial compartment of the lung (Riches D W H, et al., “Inflammation in the pathogenesis of interstitial lung disease”, In: Interstitial Luna Disease, Schwarz M I and King T E (editors), BC Decker Inc, Hamilton, pp: 187-220 (2003)). CC chemokines such as MCP-1 released from injured lung tissue play a fundamental role in triggering the recruitment of leukocytes cells into the lung (Strieter R M, et al., “CXC chemokines in vascular remodeling related to pulmonary fibrosis”, Am. J. Respir. Cell Mol. Biol. 29:S67-9 (2003)). Other inflammatory mediators including the pro-inflammatory cytokine TNF-α, the pro-coagulant factor thrombin and the Th2 cytokine IL-13 may also favor the migration of inflammatory cells into the lung by stimulating the secretion of chemoattractant proteins from injured lung resident cells (Strieter et al., ibid). In addition, exaggerated release of enzymes such as elastase and myeloperoxidase (MPO) from activated leukocytes may further exacerbate the lung inflammatory response (Riches et al., supra). Lung injury causes increased permeability of alveolar epithelium and vascular endothelium resulting in extravasation of plasma proteins, activation of coagulation system and deposition of fibrin clots in the alveolar spaces. In the present study, the circulating level of LDH, the BALF levels of total protein, MCP-1, TNF-1α and neutrophil-derived enzymes (elastase, MPO) were significantly decreased in TAFI (−/−) mice as compared to TAFI (+/+) and TAFI (+/−) mice. These observations suggest that TAFI deficiency protects the lung from inflammation. In the present study, histochemical studies disclosed decreased lung deposition of fibrin in TAFI (−/−) mice as compared to TAFI (+/+) and TAFI (+/−) mice. This finding may depend on lower thrombin generation and higher fibrinolytic activity in the lungs from mice with TAFI deficiency as demonstrated by the decreased level of TAT and elevated values of urokinase/TAT ratio in BALF from TAFI (+/−) mice as compared to that from their wild type counterparts. Thus, lower fibrin deposition, either due to lower formation or faster clearnce is the most probable explanation for the lower inflammatory reaction in lungs from TAFI deficient mice.

Cytokines released from helper T cells play a critical role in the pathogenesis of lung injury and fibrosis (Agostini C, et al., “Immune effector cells in idiopathic pulmonary fibrosis”, Curr. Opin. Pulm. Med. 3:348-55 (1997)). Two functionally distinct subsets of helper T cells have been differentiated depending on their cytokine expression profile: (1) Th1, which mainly secretes IFN-γ and interleukin (IL)-2, and (2) Th2, which produces IL-4, IL-5, IL-13, IL-6 and IL-10 (Keane M P, et al., “Cytokine biology and the pathogenesis of interstitial lung disease”. In: Interstitial Lung Disease, Schwarz M I and King T E (editors), BC Decker Inc, Hamilton, p: 245-275 (2003)). An imbalance between Th1/Th2 cells occurs in lung fibrosis, with the balance tipped away from the normally predominant Th1 cells in favor of Th2 cells. A localized Th2 response by the host leads to excessive fibrosis, whereas a predominant Th1 response protects the host from an exuberant fibrotic response (Keane et al., ibid).

The hallmark of bleomycin-induced lung fibrosis is the extensive deposition of collagen in the interstitial and alveolar spaces of the lung (Chapman, supra). In this study, although all groups of bleomycin-treated mice developed lung fibrosis compared to saline-treated mice, enhanced fibrotic lesion development was observed in bleomycin-treated TAFI (+/+) and TAFI (+/−) mice relative to TAFI (−/−) mice. In addition, the total lung collagen content and the BALF level of soluble collagen were significantly increased in bleomycin-treated TAFI (+/+) and TAFI (+/−) mice as compared to bleomycin-treated TAFI (−/−) mice. The lung tissue total collagen content in TAFI (−/−) was approximately one-half of that observed in wild type and heterozygous mice. There are several possible explanations for the relative protection of TAFI (−/−) mice from lung fibrosis. While not wishing to be bound to any particular mechanism of action, one explanation for the relative protection of TAFI deficient mice from lung fibrosis may be that the inflammatory response is attenuated with subsequent reduced expression of pro-fibrotic factors in the lung. In agreement with this, in the present study, the BALF concentration of PDGF and TGF-β1 was significantly decreased in TAFI (−/−) mice as compared to that observed in their heterozygous and wild type counterparts. Another explanation for the protection of TAFI deficiency from lung fibrosis may be the high fibrinolytic activity in TAFI (−/−) mice; the BALF fibrinolytic activity in both TAFI (+/+) and TAFI (+/−) mice was one-half of that observed in TAFI (−/−) mice. Although it has been recently demonstrated that, intraalveolar fibrin is not required for lung fibrosis it can promote it by providing a provisional matrix onto which fibroblasts migrate and produce collagens (Ploplis V A, et al., “A total fibrinogen deficiency is compatible with the development of pulmonary fibrosis in mice”, Am. J. Pathol. 157:703-8 (2000)). In addition, fibrin-independent effects of plasmin such as its stimulation of pro-metalloproteinase activation and secretion of hepatocyte growth factor may also attenuate lung fibrosis in TAFI-deficient mice (Murphy G, et al., “Mechanisms for pro matrix metalloproteinase activation”, APMIS.107:38-44 (1999); Hattori N, et al., “The plasminogen activation system reduces fibrosis in the lung by a hepatocyte growth factor-dependent mechanism”, Am. J. Pathol. 164:1091-8 (2004)). Thus, the high fibrinolytic activity may be an important mechanism for the decreased development of lung fibrosis in TAFI deficient mice observed in our study.

In conclusion, the results reported in this study showed that TAFI deficiency is associated with an attenuated inflammatory response and lower collagen deposition in the lung, suggesting that the antifibrinolytic activity of TAFI is involved in the pathogenesis of lung fibrosis.

### EXAMPLES

### MATERIALS AND METHODS

Animals

TAFI (+/+), TAFI (+/−) and TAFI (−/−) mice in a mixed background of C57BL/6 and 129/Sv strains were developed and characterized as previously described (Nagashima, M, et al., “Thrombin-activatable fibrinolysis inhibitor (TAFI) deficiency is compatible with murine life”, J. Clin. Invest. 109:101-10 (2002)). Mice were maintained on a constant 12-hour light/12-hour dark cycle in a temperature- and humidity-controlled room and were given ad libitum access to food and water. In all experiments, wild-type littermates were used as controls. Female mice weighing 18-22 g and at an age between 8 and 12 weeks were used in the experiments. The Mie University's Committee on Animal Investigation approved the animal protocol.

Animal Model of Lung Fibrosis

Lung damage was elicited by administering bleomycin (BLM) (Nihon Kayaku, Tokyo, Japan) at a dose of 100 mg/kg by constant subcutaneous infusion from osmotic minipumps (model 2001; Alza Corp., Palo Alto, Calif.) as previously described (Yasui et al., supra). BLM was dissolved in sterile saline and loaded into 7-day minipumps. The control animals were treated similarly except that the minipumps contained sterile saline. Before pump implantation, the mice were anesthetized with intraperitoneal sodium pentobarbital at a dose of 62.5 mg/kg of body weight. The minipump was implanted through a small incision in the back of the mouse, and the wound was then sealed with wound clips. At the end of the experiment, the pumps were examined to determine if they had delivered the entire dosage of their contents to each mouse.

Experimental Design

Four groups of animals were included in the experiments (each group with n=12): TAFI (+/+) mice treated with saline (WT/SAL) or BLM (WT/BLM), TAFI (+/−) treated with BLM (HETE/BLM) and TAFI (−/−) treated with BLM (KO/BLM).

Bronchoalveolar Lavage and Blood Sampling

All animals were sacrificed on day 21 after BLM or saline subcutaneous infusion by intraperitoneal injection of pentobarbital to take samples for biochemical and histological examinations. Blood samples were collected by heart puncture and placed in tubes containing 1/10 volume of 3.8% sodium citrate. Then, the trachea was cannulated with polyethylene tubing attached to a 20-gauge needle on a tuberculin syringe, and BALF was obtained by per tracheal infusion of 0.75 ml of isotonic saline two times. The recovery of BALF ranged between 1.4 and 1.5 mL with no significant differences in the volume recovered between control and treated mice. The recovered fluid was filtered through a single layer of gauze to remove mucus. BALF was then centrifuged at 1000× g for 10 min at 4° C. and the cell-free supernatant was aliquoted and stored immediately at 80° C. until use.

Measurement of Total Soluble Collagen

Total soluble collagen was measured in BALF samples by the Sircol assay (Biocolor, Belfast, N. Ireland). One milliliter of Sirius Red reagent was mixed with 75 μL of BALF sample for 30 min at room temperature. The collagen-dye complex was precipitated by centrifugation at 16,000×g for 5 min, dissolved in 1 mL 0.5 M NaOH, and then the absorbance was measured at 540 nm. The amount of soluble collagen was determined by comparison with a standard curve provided by the manufacturer. The assay was performed in duplicate, and the mean calculated for each individual sample.

Biochemical Analysis

The concentration of total protein in BALF was measured using a dye-binding assay (Bio-Rad Laboratories, Hercules, Calif.) following the manufacturer's instructions. The plasma level of lactic dehydrogenase (LDH) was measured using a commercial kit (LDH ICII kit, Wako Pure Chemical industry, Osaka, Japan) following the manufacturer's instructions. The BALF and plasma concentrations of thrombin-antithrombin complex (TAT) were measured using an enzyme immunoassay (EIA) kit from Cedarlane Laboratories (Ontario, Canada). The concentrations of monocyte chemoattractant protein-1 (MCP-1), tumor necrosis factor (TNF)-α and transforming growth factor (TGF)-β1 in BALF were measured using commercial EIA kits from BD Biosciences Pharmigen (San Diego, Calif.). The Th1 cytokine IFN-γ (Biosource International, Amarillo, Calif.) and the Th2 cytokine IL-13 (Genzyme, Minneapolis, Minn.) were measured using commercial immunoassay kits. The inter-assay and intra-assay coefficients of variability of all the cytokine kits were less than 10%. The BALF level of total platelet-derived growth factor (PDGF) was measured using polyclonal anti-PDGF (Genzyme, Boston, Mass.) and biotin-labeled anti-PDGF antibodies. Values of PDGF were extrapolated from a curve created using standard concentrations of PDGF antigen. The inter- and intra-assay coefficients of variation were less than 5%. The urokinase activity in BALF was determined spectrophotometrically using a specific substrate (S-2444). Values of urokinase activity were extrapolated from a curve created using standard concentrations of urokinase antigen. The inter- and intra-assay coefficients of variation were less than 10%.

Neutrophil Elastase Activity in BALF

The activity of neutrophil elastase in BALF was measured spectrophotometrically using the synthetic substrate methoxysuccinyl-ala-ala-pro-val-paranitroanilide (MeOSAAPVpNa) (Sigma Chemical (St Louis, Mo.). Twenty microliters of standard or sample were added to a 96-well microtiter plate followed by MeOSAAPVpNa (0.3 mM) in phosphate buffer (0.2 M, pH 8.0). The mixture was incubated for 1 hr at 37° C. and then the reaction was stopped by the addition of 200 μL of 1 N acetic acid. The absorbance was read at 405 nM and the activity of elastase was calculated from a standard curve by interpolation. The interassay coefficient of variation was less than 2% with a lower limit of detection of 1 nM.

Myeloperoxidase Assay

Myeloperoxidase activity in BALF was spectrophotometrically measured. Briefly, 50 μL of BALF samples and standards were applied to a 96-well plate and then 100 μL of assay solution, which was prepared by mixing 1 mL of the peroxidase substrate 2,2′-azino-bis(ethylbenzylthiazoline-6-sulfonic acid) diammonium salt, 50 μL of citrate phosphate buffer, and 5 μL of 30% hydrogen peroxide (BDH/Merck). The reaction was allowed to develop at room temperature before the optical density at 405 nm was measured with a microplate reader. Each sample was assayed in duplicate wells.

Histological Study

Lung samples for histological examination were drawn on day 21 after starting BLM or saline subcutaneous infusion. After thoracotomy, the pulmonary circulation was flushed with saline and the lungs were removed. The left lung of the mouse was perfused with 10% neutral buffered formalin and fixed in formalin for 24 hr. The tissue sections were embedded in paraffin and then prepared for hematoxilin/eosin and Mallory-Azan staining.

Immunohistochemistry

Five-micron-thick paraffin sections layered on silane-coated slides were used for immunohistochemical staining of collagen type I and fibrin using primary rabbit anti-mouse collagen I (Bethyl Laboratories, Montgomery, Tex.) and anti-mouse fibrin(ogen) (DAKO, Denmark) antibodies. Before immunostaining using the RTU Vectastain Kit (Burlingame, Calif.), endogeneous peroxidase in the tissue was blocked by incubation using 1% H₂O₂ in PBS, pH 7.4. Nonspecific binding was blocked with 10% goat serum in phosphate-buffered saline (PBS). The histological sections were then incubated for 1 hr at 37° C. with 1 μg/mL of each primary antibody. The samples were then treated successively with biotin-labeled rabbit anti-mouse IgG, peroxidase-labeled streptavidin and peroxidase substrate using the Catalyzed Signal Amplification System from Dako. The sections were counterstained with hematoxilin. Controls for immunospecificity which were included in all experiments by matching concentrations of non-immune normal rabbit serum were all negative (data not shown).

Statistical Analysis

All data are expressed as the mean ± standard error (S.E.) unless otherwise specified. The difference between three or more variables was calculated by analysis of variance. Statistical analyses were carried out using the StatView 4.1 package software for the Macintosh (Abacus Concepts, Berkeley, Calif.).

### RESULTS

Inflammatory Response and Lung Injury

As markers of lung inflammation, the concentration of total protein in BALF and that of LDH in plasma were measured in each group of animals. The BALF concentration of total protein and the circulating level of LDH were markedly increased in WT/BLM, HETE/BLM and KO/BLM as compared to WT/SAL group. However, the concentrations of both total protein and LDH were significantly lower in KO/BLM than in WT/BLM and HETE/BLM groups. The inflammatory response in the injured lung tissue is characterized by accumulation of inflammatory cells particularly of neutrophils, which secrete several proteases including elastase and MPO. In this study, the BALF concentrations of both elastase and MPO were significantly increased in WT/BLM, HETE/BLM mice as compared to WT/SAL group, but the levels of both markers were significantly lower in KO/BLM mice than in WT/BLM or HETE/BLM mice. The BALF concentration of elastase but not that of MPO was significantly increased in KO/BLM as compared to WT/SAL group. Overall, these findings suggest that TAFI deficient mice are partially protected from lung injury as compared to their wild and heterozygous type counterparts.

Cytokine Expression

The cytokine profile of the immune/inflammatory response may determine the disease phenotype responsible for either resolution or progression to end-stage fibrosis. Th1 cytokines such as IFN-γ exerts suppressive effect on production of extracellular matrix, while Th2 cytokines such IL-13 stimulate the secretion of collagen type-I and type III from fibroblasts (Riches et al., supra). In the present study, the level of IL-13 and the IL-13/IFN-γ ratio were increased but the level of IFN-γ was decreased in BALF from WT/BLM, HETE/BLM and KO/BLM mice as compared to WT/SAL mice. Neither the BALF level of IL-13 nor that of IFN-γ was significantly different between KO/BLM and WT/BLM and HETE/BLM mice. The chemokine MCP-1 and the pro-inflammatory cytokine TNF-α have been also implicated in the pathogenesis of lung fibrosis for promoting the infiltration of monocytes and macrophages and for stimulating the proliferation of mesenchymal cells (Riches et al., supra). In the present study, the BALF concentrations of MCP-1 and TNF-α were significantly increased in WT/BLM and HETE/BLM animal groups, but not in the KO/BLM group, as compared to WT/SAL mice. However, the levels of both MCP-1 and TNF-α were markedly lower in KO/BLM than in WT/BLM and HETE/BLM mice. In addition, the BALF levels of MCP-1 and TNF-α were not significantly different between KO/BLM and WT/SAL groups. These observations suggest that TAFI deficiency affects lung inflammation not through modulating the Th2/Th1 ratio but through the suppression of other pro-inflammatory mediators such as MCP-1 or TNF-α.

Activation of Coagulation and Fibrinolysis Systems

Activation of coagulation system and hypofibrinolysis in the intraalveolar space plays a fundamental role in the development of lung fibrosis (Swaisgood et al., supra). We measured the level of TAT as a marker of coagulation system activation, and the ratio of urokinase activity to TAT as a marker of fibrinolysis activity. The BALF level of TAT was significantly increased in WT/BLM, HETE/BLM and KO/BLM mice as compared to WT/SAL mice, but it was significantly lower in KO/BLM and HETE/BLM mice than in WT/BLM mice. On the other hand, the fibrinolytic activity as measured by the ratio of urokinase to TAT was significantly decreased in both WT/BLM and HETE/BLM groups as compared to WT/SAL group. The urokinase/TAT ratio was significantly lower in KO/BLM mice than in WT/SAL mice but it was markedly higher than in the WT/BLM and HETE/BLM groups. These observations suggest that TAFI deficiency is associated with an increase in fibrinolytic activity in the lung.

Growth Factor Expression

TGE-β1 promotes extracellular matrix deposition by enhancing the synthesis and secretion of collagens and of tissue-type metalloproteinase inhibitors (Riches et al., supra). PDGF is a potent mitogen and chemoattractant for mesenchymal cells and also, it is able to induce gene expression of cell matrix-related molecules such as collagen, fibronectin and glycosaminoglycans (Riches et al., supra). In the present study, the BALF concentrations of both TGF-β1 and PDGF were significantly increased in the WT/BLM and HETE/BLM groups, but not in the KO/BLM group, as compared to WT/SAL mice. In addition, both growth factors were markedly reduced in KO/BLM mice as compared to WT/BLM and HETE/BLM mice. No difference was found between KO/BLM and WT/SAL groups. These results suggest that TAFI deficiency is associated with lower secretion of growth factors in the lung.

Total Lung Content of Collagen

Collagen deposition in the lung was assessed by measuring the collagen content in lung tissue and in BALF from each group of animals. Compared to the WT/SAL group, the collagen content of lung tissue and the BALF level of soluble collagen were significantly increased in WT/BLM, HETE/BLM and KO/BLM groups. However, the lung tissue content and BALF level of collagen were significantly lower in KO/BLM mice than in both WT/BLM and HETE/BLM groups. These findings suggest the protective role of TAFI deficiency in lung fibrosis.

Correlation of Variables in BALF from Bleomycin-Treated Mice.

To uncover factors involved in the process of lung fibrosis in our animal model, the relationship between variables was evaluated in BALF from animals treated with bleomycin. The concentration of soluble collagen, a marker of collagen deposition and synthesis was proportionally and significantly correlated with the concentrations of IL-13 (r=0.4, p<0.01), IL-13/IFN-γ ratio (r=0.4, p<0.05) and TAT (r=0.5, p<0.01) and involvement of Th2 with the ratio of urokinase to TAT (r=0.4, P0.01), suggesting the involvement of Th2 cytokines, activation of coagulation and fibrinolytic activity in the process of lung fibrosis. The urokinase/TAT ratio was also significantly correlated with MPO and TNF-α, suggesting the possible detrimental effect of these factors on fibrinolytic activity in our animal model.

Histologic Findings

On day 21, compared to the KO/BLM group, animals of the WT/BLM and HETE/BLM groups showed severe fibrotic changes in the lungs, expanding to the central regions of the lung parenchyma, involving the perivascular and peribronchiolar areas, and with more uniform areas of consolidation in subpleural regions of the lung.

Immunohistochemical Findings

Deposition of collagen was significantly more prominent in mice of the WT/BLM and HETE/BLM groups as compared to those from the KO/BLM group. Immunostaning of lung tissue from mice of the WT/BLM and HETE/BLM groups showed significantly more deposition of extracellular collagen type I in thickened alveolar as compared to the KO/BLM group. Similarly, fibrin immunostaining disclosed more fibrin formation in the interstitial and alveolar spaces in WT/BLM and HETE/BLM than in KO/BLM mice. These observations suggest that low fibrin formation is associated with less collagen deposition in TAFI-deficient mice.

What is claimed is:

1. A method of treating pulmonary fibrosis comprising administering a therapeutically effective amount of a compound of formula (I) to a patient in need thereof: 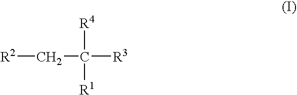
wherein:
R¹ is hydrogen, alkyl, alkenyl, aralkyl, or aralkenyl;
R² is —SH, —S—C(O)—R⁸, —P(O)(OR⁵)₂, —P(O)(OR⁵)R⁶, —P(O)(OR⁵)—R⁷—N(R⁶)₂, —P(O)(OR⁵)—R⁷—C(O)—R⁸, —P(O)(OR⁵)—R⁷—N(R⁵)—C(O)OR⁸, —P(O)(OR⁵)—R⁷—N(R⁵)—C(O)—R⁷—N(R⁵)—C(O)OR⁸, —P(O)(OR⁵)—R⁷—N(R⁵)—S(O)₂—R⁹, or —P(O)(OR⁵)—R⁷—N(R⁵)—C(S)—N(R⁶)₂;
R³ is tetrazole, —C(O)OR⁶, —C(O)O—R⁷—OC(O)R⁵, —S(O)OR⁵, —S(O)₂OR⁵, —P(O)(OR⁵)₂, —P(O)(OR⁵)R⁶, or —B(OR⁵)₂;
R⁴ is aryl optionally substituted by one or more substituents selected from the group consisting of alkyl, halo, haloalkyl, haloalkoxy, mercapto, alkylthio, phenyl, cycloalkyl, nitro, cyano, —OR⁶, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is —R⁷ N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR , —C(O)N(R⁶)₂ or —N(R⁵)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶);
provided that when R³ is —C(O)OH or when R⁴ is a substituted aryl or substituted N-heterocyclyl, R² can not be —P(O)(OR⁵)—R⁷—N(H)—C(O)OR⁸ or —P(O)(OR⁵)—R⁷—N(H)—C(O)—R⁷—N(R⁵)—C(O)OR⁸;
as a single stereoisomer, a mixture of stereoisomers, or as a racemic mixture of stereoisomers; or a pharmaceutically acceptable salt thereof.

2. The method of claim 1 wherein:
R¹ is hydrogen;
R² is —SH or —S—C(O)—R⁸;
R³ is tetrazole, —C(O)OR⁶ or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or —C(O)N(R⁶)₂; and
each R⁸ is independently alkyl, alkenyl, aryl, aralkyl or aralkenyl.

3. The method of claim 2 wherein:
R¹ is hydrogen;
R² is —SH or —S—C(O)—R⁸;
R³ is —C(O)OR⁶;
R⁴ is aryl optionally substituted by one or more substituents selected from the group consisting of halo, nitro, —N(R⁶)₂, —R⁷—N(R⁶)₂ and —N(R⁵)—C(NR⁵)—N(R⁵)₂;
each R⁵ is independently hydrogen, alkyl or aralkyl;
each R⁶ is independently hydrogen, alkyl, aryl or aralkyl;
R⁷ is a straight or branched alkylene chain; and
R⁸ is alkyl, alkenyl, aryl, aralkyl or aralkenyl.

4. The method of claim 3 wherein the compound of formula (I) is selected from the group consisting of:
2-(4-guanidinophenyl)-3-mercaptopropanoic acid;
2-(3-guanidinophenyl)-3-mercaptopropanoic acid;
2-(3-aminophenyl)-3-mercaptopropanoic acid; and
2-(2-chloro-5-guanidinophenyl)-3-mercaptopropanoic acid.

5. The method of claim 2 wherein:
R¹ is hydrogen;
R² is —SH, or —S—C(O)—R⁸;
R³ is —C(O)OR⁶;
R⁴ is 3(4)-piperidinyl wherein the nitrogen atom in the piperidinyl radical is optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, aryl or aralkyl;
R⁷ is a straight or branched alkylene chain optionally substituted by hydroxy, mercapto, alkylthio, aryl, cycloalkyl, —N(R⁶)₂, —C(O)OR⁶, or —C(O)N(R⁶)₂; and
R⁸ is independently alkyl, alkenyl, aryl, aralkyl or aralkenyl.

6. The method of claim 5 wherein the compound of formula (I) is selected from the group consisting of:
2-(piperidin-4-yl)-3-mercaptopropanoic acid;
2-(1-amidinopiperidin-4-yl)-3-mercaptopropanoic acid;
2-(1-(1-iminoethyl)piperidin4-yl)-3-mercaptopropanoic acid;
2-(1-(aminomethylcarbonyl)piperidin4-yl)-3-mercaptopropanoic acid;
2-(piperidin-3-yl)-3-mercaptopropanoic acid; and
2-(1-amidinopiperidin-3-yl)-3-mercaptopropanoic acid.

7. The method of claim 1 wherein:
R¹ is hydrogen;
R² is —P(O)(OR⁵)₂, —P(O)(OR⁵)R⁶ or —P(O)(OR⁵)—R⁷—C(O)—R⁸;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or —C(O)N(R⁶)₂; and
each R⁸ is independently alkyl, alkenyl, aryl, aralkyl or aralkenyl.

8. The method of claim 7 wherein:
R¹ is hydrogen;
R² is —P(O)(OR⁵)₂, —P(O)(OR⁵)R⁶ or —P(O)(OR⁵)—R⁷—C(O)—R⁸;
R³ is —C(O)OR⁶;
R⁴ is aryl optionally substituted by one or more substituents selected from the group consisting of halo, nitro, —N(R⁶)₂, —R⁷—N(R⁶)₂ and —N(R⁵)—C(NR⁵)—N(R⁵)₂;
each R⁵ is independently hydrogen, alkyl or aralkyl;
each R⁸ is independently hydrogen, alkyl, aryl or aralkyl;
each R⁷ is independently a straight or branched alkylene chain optionally substituted by aryl, —N(R⁶)₂ or —C(O)OR⁶; and
R⁸ is alkyl, alkenyl, aryl, aralkyl or aralkenyl.

9. The method of claim 8 wherein the compound of formula (I) is selected from the group consisting of:
2-(3-guanidinophenyl)-3-phosphonopropanoic acid;
2-(3-aminophenyl)-3-((phenyl)(hydroxy)phosphinoyl)propanoic acid;
2-(3-aminophenyl)-3-((4-phenylbutyl)(hydroxy)phosphinoyl)propanoic acid;
2-(3-aminophenyl)-3-((pentyl)(hydroxy)phosphinoyl)propanoic acid;
2-(3-guanidinophenyl)-3-((phenyl)(hydroxy)phosphinoyl)propanoic acid;
2-(3-guanidinophenyl)-3-((4-phenylbutyl)(hydroxy)phosphinoyl)propanoic acid;
2-(3-guanidinophenyl)-3-((pentyl)(hydroxy)phosphinoyl)propanoic acid;
2-(3-guanidinophenyl)-3-((4-methylpentyl)(hydroxy)phosphinoyl)propanoic acid;
2-(3-guanidinophenyl)-3-((3-phenylpropyl)(hydroxy)phosphinoyl)propanoic acid;
2-(3-guanidinophenyl)-3-((3-phenylprop-2-enyl)(hydroxy)phosphinoyl)propanoic acid;
2-(3-guanidinophenyl)-3-((phenylmethyl)(hydroxy)phosphinoyl)propanoic acid;
2-(3-guanidinophenyl)-3-((pentyl)(hydroxy)phosphinoyl)propanoic acid methyl ester;
2-(3-guanidinophenyl)-3-((ethyl)(hydroxy)phosphinoyl)propanoic acid;
2-(3-guanidinophenyl)-3-((2-phenylethyl)(hydroxy)phosphinoyl)propanoic acid; and
2-(3-guanidinophenyl)-3-((2-(methylcarbonyl)ethyl)(hydroxy)phosphinoyl)propanoic acid.

10. The method of claim 1 wherein:
R¹ is hydrogen;
R² is —P(O)(OR⁵)—R⁷—N(R⁵)—C(O)OR⁸;
R³ is —C(O)OR⁶ (where R⁶ is alkyl, aryl or aralkyl);
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂—N(R⁵)—C(O)—N(R⁶)₂ and —N(R⁵)—C(O)—R⁷—N(R⁶₂) where each R⁶ is independently hydrogen, alkyl, aryl or aralkyl;
each R⁵ is independently hydrogen, alkyl or aralkyl;
each R⁷ is a straight or branched alkylene chain optionally substituted by aryl, —N(R⁶)₂ or —C(O)OR⁶; and
each R⁸ is independently alkyl, alkenyl, aryl, aralkyl or aralkenyl.

11. The method of claim 10 wherein the compound of formula (I) is selected from the group consisting of:
2-(3-(t-butoxycarbonylamino)methylphenyl)-3-((1-(benzyloxycarbonyl)amino-2-methylpropyl)(hydroxy)phosphinoyl)propanoic acid t-butyl ester; and
2-(3-(t-butoxycarbonylamino)methylphenyl)-3-((1-(benzyloxycarbonyl)amino-2-methylpropyl)(ethoxy)phosphinoyl)propanoic acid t-butyl ester.

12. The method of claim 1 wherein:
R¹ is hydrogen;
R² is —P(O)(OR⁵)—R⁷—N(R⁶)₂ or —P(O)(OR⁵)—R⁷—N(R⁵)—C(S)—N(R⁶)₂;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
each R⁵ is independently hydrogen, alkyl or aralkyl;
each R⁶ is independently hydrogen, alkyl, alkenyl, alkynyl, aryl, aralkyl or aralkenyl;
each R⁷ is a straight or branched alkylene chain optionally substituted by hydroxy, mercapto, alkylthio, aryl, cycloalkyl, —N(R⁶)₂, —C(O)OR⁶, or —C(O)N(R⁶)₂; and
each R⁸ is independently alkyl, alkenyl, aryl, aralkyl or aralkenyl.

13. The method of claim 12 wherein the compound of formula (I) is selected from the group consisting of:
2-(3-(t-butoxycarbonylamino)methylphenyl)-3-((1-amino-2-methylpropyl)(hydroxy)-phosphinoyl)propanoic acid t-butyl ester; and
2-(3-(t-butoxycarbonylamino)methylphenyl)-3-((1-amino-2-methylpropyl)(ethoxy)-phosphinoyl)propanoic acid t-butyl ester.

14. The method of claim 1 wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is —R⁷N(R⁵)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

15. The method of claim 14 wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain (optionally substituted by hydroxy, mercapto, alkylthio, aryl, cycloalkyl, —N(R⁶)₂, —C(O)OR⁶, or —C(O)N(R⁶)₂),
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

16. The method of claim 15 wherein:
R¹ is hydrogen;
R² is —P(O)(OR⁵)—R⁷—N(R⁵)—S(O)₂—R⁹;
R³ is tetrazole, —C(O)OR, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or —C(O)N(R⁶)₂,
each R⁸ is independently alkyl, alkenyl, aryl, aralkyl or aralkenyl; and
R⁹ is alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

17. The method of claim 16 wherein the compound of formula (I) is selected from the group consisting of:
2-(3-(amino)methylphenyl)-3-((1-(3-phenylpropylsulfonyl)amino-2-methylpropyl)-(hydroxy)phosphinoyl)propanoic acid, methyl ester;
2-(3-(t-butoxycarbonylamino)methylphenyl)-3-((1-(3-phenylpropylsulfonyl)amino-2-methylpropyl)(hydroxy)phosphinoyl)propanoic acid;
2-(3-(t-butoxycarbonylamino)methylphenyl)-3-((1-(3-phenylpropylsulfonyl)amino-2-methylpropyl)(hydroxy)phosphinoyl)propanoic acid, methyl ester;
2-(3-(amino)methylphenyl)-3-((1-(3-phenylpropylsulfonyl)amino-2-methylpropyl)-(hydroxy)phosphinoyl)propanoic acid;
(2R)-2-(3-(amino)methylphenyl)-3-(((1R)-1-(3-phenylpropylsulfonyl)amino-2-methylpropyl)(hydroxy)phosphinoyl)propanoic acid;
(2S)-2-(3-(amino)methylphenyl)-3-(((1R)-1-(3-phenylpropylsulfonyl)amino-2-methylpropyl)(hydroxy)phosphinoyl)propanoic acid;
(2R/S)-2-(3-(amino)methylphenyl)-3-(((1S)-1-(3-phenylpropylsulfonyl)amino-2-methylpropyl)(hydroxy)phosphinoyl)propanoic acid;
(2R/S)-2-(3-(amino)methylphenyl)-3-(((1R)-1-(3-phenylpropylsulfonyl)amino-2-methylpropyl)(hydroxy)phosphinoyl)propanoic acid;
(2R)-2-(3-(amino)methylphenyl)-3-(((1S)-1-(3-phenylpropylsulfonyl)amino-2-methylpropyl)(hydroxy)phosphinoyl)propanoic acid;
(2S)-2-(3-(amino)methylphenyl)-3-(((1S)-1-(3-phenylpropylsulfonyl)amino-2-methylpropyl)(hydroxy)phosphinoyl)propanoic acid;
2-(3-(t-butoxycarbonylamino)methylphenyl)-3-((1-(3-phenylpropylsulfonyl)amino-2-methylpropyl)(ethoxy)phosphinoyl)propanoic acid, t-butyl ester;
2-(3-(amino)methylphenyl)-3-((1-(2-phenylethylsulfonyl)amino-2-methylpropyl)-(hydroxy)phosphinoyl)propanoic acid;
2-(3-(amino)methylphenyl)-3-((1-(benzylsulfonyl)amino-2-methylpropyl)-(hydroxy)phosphinoyl)propanoic acid;
2-(3-(amino)methylphenyl)-3-((1-(2-(naphth-1-yl)ethylsulfonyl)amino-2-methylpropyl)-(hydroxy)phosphinoyl)propanoic acid;
2-(3-(amino)methylphenyl)-3-((1-(3-(4-methoxyphenyl)propylsulfonyl)amino-2-methylpropyl)(hydroxy)phosphinoyl)propanoic acid;
2-(3-(amino)methylphenyl)-3-((1-(2-(4-methoxyphenyl)ethylsulfonyl)amino-2-methylpropyl)(hydroxy)phosphinoyl)propanoic acid;
2-(3-(amino)methylphenyl)-3-((1-(methylsulfonyl)amino-2-methylpropyl)(hydroxy)phosphinoyl)propanoic acid;
2-(3-(amino)methylphenyl)-3-((1-(2-benzyloxyethylsulfonyl)amino-2-methylpropyl)-(hydroxy)phosphinoyl)propanoic acid;
2-(3-(amino)methylphenyl)-3-((1-(2-hydroxyethylsulfonyl)amino-2-methylpropyl)-(hydroxy)phosphinoyl)propanoic acid;
2-(3-aminophenyl)-3-((1-(3-phenylpropylsulfonyl)amino-2-methylpropyl)-(hydroxy)phosphinoyl)propanoic acid;
2-(3-guanidinophenyl)-3-((1-(3-phenylpropylsulfonyl)amino-2-methylpropyl)(hydroxy)-phosphinoyl)propanoic acid;
2-(3-(amino)methylphenyl)-3-((1-(4-phenylbutylsulfonyl)amino-2-methylpropyl)-(hydroxy)phosphinoyl)propanoic acid, and
2-(3-(amino)methylphenyl)-3-((1-(2-phenylethenylsulfonyl)amino-2-methylpropyl)-(hydroxy)phosphinoyl)propanoic acid.

18. The method of claim 15 wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain optionally substituted by hydroxy, mercapto, alkylthio, aryl, cycloalkyl, —N(R⁶)₂, —C(O)OR⁶, or —C(O)N(R⁶)₂,
each R⁸ is independently alkyl, alkenyl, aryl, aralkyl or aralkenyl; and
R⁹ is aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

19. The method of claim 18 wherein the compound of formula (I) is selected from the group consisting of:
2-(3-(amino)methylphenyl)-3-((1-(naphth-1-ylsulfonyl)amino-2-methylpropyl)(hydroxy)-phosphinoyl)propanoic acid;
2-(3-(amino)methylphenyl)-3-((1-(3-trifluoromethylphenylsulfonyl)amino-2-methyl-propyl)(hydroxy)phosphinoyl)propanoic acid;
2-(3-(amino)methylphenyl)-3-((1-(4-pentylphenylsulfonyl)amino-2-methylpropyl)(hydroxy)phosphinoyl)propanoic acid;
2-(3-(amino)methylphenyl)-3-((1-(4-acetamidophenylsulfonyl)amino-2-methylpropyl)(hydroxy)phosphinoyl)propanoic acid;
2-(3-(amino)methylphenyl)-3-((1-(4-phenylphenylsulfonyl)amino-2-methylpropyl)-(hydroxy)phosphinoyl)propanoic acid; and
2-(3-(amino)methylphenyl)-3-((1-(phenylsulfonyl)amino-2-methylpropyl)(hydroxy)-phosphinoyl)propanoic acid.

20. The method of claim 15 wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, —N(R⁶)—C(O)—R⁶, —R⁷—N(R⁶)—C(O)—R⁶, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain (optionally substituted by hydroxy, mercapto, alkylthio, aryl, cycloalkyl, —N(R⁶)₂, —C(O)OR⁶, or —C(O)N(R⁶)₂),
each R⁸ is independently alkyl, alkenyl, aryl, aralkyl or aralkenyl; and
R⁹ is —R⁷—N(R⁶)—C(O)OR⁸.

21. The method of claim 20 wherein the compound of formula (I) is 2-(3-(amino)methylphenyl)-3-((1-(3-phenyl-2-(benzyloxycarbonyl)aminopropylsulfonyl)-amino-2-methylpropyl)(hydroxy)phosphinoyl)propanoic acid.

22. The method of claim 15 wherein:
R¹ is hydrogen;
R² is —P(O)(OR⁵)—R⁷—N(R⁵)—S(O)₂—R⁹;
R³ is tetrazole, —C(O)OR⁶, or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸; —R⁷—N(R⁶)—C(O)OR⁸, —N(R⁶)—C(O)—R⁶, —R⁷—N(R⁶)—C(O)—R⁶, and —N(R⁵)—C(NR⁵)—N(R⁵)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently a straight or branched alkylene chain (optionally substituted by hydroxy, mercapto, alkylthio, aryl, cycloalkyl, —N(R⁶)₂, —C(O)OR⁶, or —C(O)N(R⁶)₂),
each R⁸ is independently alkyl, alkenyl, aryl, aralkyl or aralkenyl; and
R⁹ is N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

23. The method of claim 22 wherein the compound of formula (I) is selected from the group consisting of:
2-(3-(amino)methylphenyl)-3-((1-(thien-2-ylsulfonyl)amino-2-methylpropyl)-(hydroxy)phosphinoyl)propanoic acid; and
2-(3-(amino)methylphenyl)-3-((1-(benzothiadiazolylsulfonyl)amino-2-methylpropyl)-(hydroxy)phosphinoyl)propanoic acid.

24. The method of claim 1 wherein:
R¹ is hydrogen;
R² is —P(O)(OR⁵)—R⁷—N(R⁵)—C(O)OR⁸;
R³ is —C(O)OR⁶;
R⁴ is unsubstituted phenyl or unsubstituted N-heterocyclyl;
each R⁵ is independently hydrogen, alkyl or aralkyl;
each R⁶ is independently hydrogen, alkyl, alkenyl, alkynyl, aryl, aralkyl or aralkenyl;
each R⁷ is a straight or branched alkylene chain optionally substituted by aryl, —N(R⁶)₂ or —C(O)OR⁶; and
R⁸ is alkyl, alkenyl, aryl, aralkyl or aralkenyl.

25. The method of claim 24 wherein the compound of formula (I) is selected from the group consisting of:
2-phenyl-3-(1-(benzyloxycarbonyl)amino-2-methylpropyl)(hydroxy)phosphinoyl)-propanoic acid; and
2-tetrahydroisoquinolinyl-3-((1-(benzyloxycarbonyl)amino-2-methylpropyl)-(hydroxy)phosphinoyl)propanoic acid.

26. A method of treating pulmonary fibrosis comprising administering a therapeutically effective amount of a compound of formula (II) to a patient in need thereof: 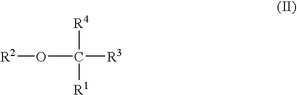
wherein:
R¹ is hydrogen, alkyl, alkenyl, aryl or aralkenyl;
R² is —P(O)(OR⁵)₂, —P(O)(OR⁵)R⁶, —P(O)(OR⁵)—R⁷—N(R⁶)₂, —P(O)(OR⁵)—R⁷—C(O)—R⁸, —P(O)(OR⁵)—R⁷—N(R⁵)—C(O)OR⁸, —P(O)(OR⁵)—R⁷—N(R⁵)—C(O)—R⁷—N(R⁵)—C(O)OR⁸, —P(O)(OR⁵)—R⁷—N(R⁵)—S(O)₂—R⁹, or —P(O)(OR⁵)—R⁷—N(R⁵)—C(S)—N(R⁶)₂;
R³ is tetrazole, —C(O))R⁶, —C(O)O—R⁷—OC(O)R⁵, —S(O)OR⁵, —S(O)₂OR⁵, —P(O)(OR⁵)₂, —P(O)(OR⁵)R⁶, or —B(OR⁵)₂;
R⁴ is aryl optionally substituted by one or more substituents selected from the group consisting of alkyl, halo, haloalkyl, haloalkoxy, mercapto, alkylthio, phenyl, cycloalkyl, nitro, cyano, —OR⁶, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR , or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶);
provided that when R³ is —C(O)OH or when R⁴ is a substituted aryl or substituted N-heterocyclyl, R² can not be —P(O)(OR⁵)—R⁷—N(H)—C(O)OR⁸ or —P(O)(OR⁵)—R⁷—N(H)—C(O)—R⁷—N(R⁵)—C(O)OR⁸;
as a single stereoisomer, a mixture of stereoisomers, or as a racemic mixture of stereoisomers; or a pharmaceutically acceptable salt thereof.

27. The method of claim 26 wherein:
R¹ is hydrogen;
R²—P(O)(OR⁵)R⁶, —P(O)(OR⁵)—R⁷—N(R⁶)₂, or —P(O)(OR⁵)—R⁷—N(R⁵)—C(S)—N(R⁶)₂;
R³ is tetrazole, —C(O)OR⁶ or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)R⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
each R⁵ is independently hydrogen, alkyl or aralkyl;
each R⁶ is independently hydrogen, alkyl, alkenyl, alkynyl, aryl, aralkyl or aralkenyl;
each R⁷ is a straight or branched alkylene chain (optionally substituted by hydroxy, mercapto, alkylthio, aryl, cycloalkyl, —N(R⁶)₂, —C(O)OR⁶, or —C(O)N(R⁶)₂); and
each R⁸ is independently alkyl, alkenyl, aryl, aralkyl or aralkenyl.

28. The method of claim 27 wherein the compound of formula (II) is selected from the group consisting of:
2-(3-guanidinophenyl)-2-((1-(2-phenylethyl)amino-2-methylpropyl)-(hydroxy)phosphinoyloxy)ethanoic acid;
2-(3-aminophenyl)-2-((phenyl)(hydroxy)phosphinoyloxy)ethanoic acid;
2-(3-guanidinophenyl)-2-((1-amino-2-methylpropyl)(hydroxy)phosphinoyloxy)ethanoic acid; and
2-(3-guanidinophenyl)-2-((1-(benzylaminothiocarbonyl)amino-2-methylpropyl)-(hydroxy)phosphinoyloxy)ethanoic acid.

29. The method of claim 26 wherein:
R¹ is hydrogen;
R² is —P(O)(OR⁵)—R⁷—N(R⁵)—S(O)₂—R⁹;
R³ is tetrazole, —C(O)OR⁶ or —C(O)O—R⁷—OC(O)R⁵;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
each R⁵ is independently hydrogen, alkyl or aralkyl;
each R⁶ is independently hydrogen, alkyl, alkenyl, alkynyl, aryl, aralkyl or aralkenyl;
each R⁷ is a straight or branched alkylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁵, —C(O)N(R⁶)₂ or —N(R⁶)C(O)R⁶), or aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

30. The method of claim 29 wherein the compound of formula (II) is selected from the group consisting of:
2-(3-guanidinophenyl)-2-((1-(benzylsulfonyl)amino-2-methylpropyl)(hydroxy)-phosphinoyloxy)ethanoic acid; and
2-(3-guanidinophenyl)-2-((1-(2-phenylethenylsulfonyl)amino-2-methylpropyl)-(hydroxy)phosphinoyloxy)ethanoic acid.

31. The method of claim 26 wherein:
R¹ is hydrogen;
R² is —P(O)(OR⁵)₂, —P(O)(OR⁵)R⁶, —P(O)(OR⁵)—R⁷—N(R⁶)₂, —P(O)(OR⁵)—R⁷—C(O)—R⁸, —P(O)(OR⁵)—R⁷—N(R⁵)—C(O)OR⁸, —P(O)(OR⁵)—R⁷—N(R⁵)—C(O)—R⁷—N(R⁵)—C(O)OR⁸, —P(O)(OR⁵)—R⁷—N(R⁵)—S(O)₂—R⁹, or —P(O)(OR⁵)—R⁷—N(R⁵)—C(S)—N(R⁶)₂;
R³ is tetrazole;
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R²), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂);
each R⁸ is independently alkyl, alkenyl, aryl, aralkyl or aralkenyl; and
R⁹ is —R⁷N(R⁶)C(O)OR⁸, haloalkyl, alkyl (optionally substituted by hydroxy, alkoxy, aralkoxy, haloalkoxy, cyano, nitro, —N(R⁶)₂, —C(O)OR⁶, —C(O)N(R⁶)₂ or —N(R⁶)C(O)R⁶), alkenyl (optionally substituted by hydroxy, alkoxy, haloalkoxy, cyano, nitro, —N(R⁶)₂, —C(O)OR⁶, —C(O)N(R⁶)₂ or —N(R⁶)C(O)R⁶), aryl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), aralkenyl (wherein the aryl group is optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶), or N-heterocyclyl (optionally substituted by alkyl, aryl, aralkyl, hydroxy, alkoxy, cyano, nitro, halo, haloalkoxy, —N(R⁶)₂, —C(O)OR⁶, —C(O)N(R⁶)₂ or —N(R⁶)C(O)R⁶).

32. The method of claim 31 wherein the compound of formula (II) is 2-methyl-1-[1-(3-guanidinophenyl)-1-tetrazolylmethoxy](hydroxy)phosphinoyl-propylcarbamic acid, benzyl ester.

33. A method of treating pulmonary fibrosis comprising administering a therapeutically effective amount of a compound of formula (III) to a patient in need thereof: 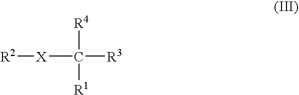
wherein:
X is —CH₂— or —O—;
R¹ is hydrogen, alkyl, alkenyl, aryl or aralkenyl;
R² is —P(O)(OR⁵)—R⁷—N(R⁵)—C(O)R⁶, —P(O)(OR⁵)—R⁷—N(R⁵)—C(O)OR⁸ or —P(O)(OR⁵)—R⁷—N(R⁵)—C(O)—R⁷—N(R⁵)—C(O)OR⁸,
R³ is —C(O)OH;
R⁴ is aryl optionally substituted by one or more substituents selected from the group consisting of alkyl, halo, haloalkyl, haloalkoxy, mercapto, alkylthio, phenyl, cycloalkyl, nitro, cyano, —OR⁶, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂);
or R⁴ is N-heterocyclyl wherein a carbon atom in the N-heterocyclyl may be optionally substituted by alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—R⁷—N(R⁶)₂, —N(R⁵)—C(NR⁵)—N(R⁵)₂, —N(R⁵)—C(O)—N(R⁶)₂ or —N(R⁵)—C(O)—R⁷—N(R⁶₂), or wherein a nitrogen atom in the N-heterocyclyl may be optionally substituted by —C(NR⁵)—N(R⁵)₂, —C(NR⁵)—R⁶, —C(O)—N(R⁶)₂ or —C(O)—R⁷—N(R⁶)₂;
each R⁵ is independently hydrogen, alkyl or aralkyl;
each R⁶ is independently hydrogen, alkyl, alkenyl, alkynyl, aryl, aralkyl or aralkenyl;
each R⁷ is independently cycloalkylene (optionally substituted by alkyl), a straight or branched alkylene chain (optionally substituted by hydroxy, mercapto, alkylthio, aryl, cycloalkyl, —N(R⁶)₂, —C(O)OR⁶, or —C(O)N(R⁶)₂), or a straight or branched alkenylene chain (optionally substituted by hydroxy, mercapto, alkylthio, aryl, cycloalkyl, —N(R⁶)₂, —C(O)OR⁶, or —C(O)N(R⁶)₂); and
each R⁸ is independently alkyl, alkenyl, aryl, aralkyl or aralkenyl;
as a single stereoisomer, a mixture of stereoisomers, or as a racemic mixture of stereoisomers; or a pharmaceutically acceptable salt thereof.

34. The method of claim 33 wherein:
X is —O—;
R² is —P(O)(OR⁵)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂).

35. The method of claim 34 wherein the compound of formula (III) is selected from the group consisting of:
2-(3-guanidinophenyl)-2-((1-(benzyloxycarbonyl)aminoethyl)(hydroxy)-phosphinoyloxy)ethanoic acid;
2-(3-guanidinophenyl)-2-(((benzyloxycarbonyl)aminomethyl)(hydroxy)-phosphinoyloxy)ethanoic acid;
2-(3-guanidinophenyl)-2-((1-(benzyloxycarbonyl)amino-2-methylpropyl)(hydroxy)phosphinoyloxy)ethanoic acid;
2-(3-guanidinophenyl)-2-((1-(benzyloxycarbonyl)aminohexyl)-(hydroxy)phosphinoyloxy)ethanoic acid;
2-(3-aminophenyl)-2-((1-(benzyloxycarbonyl)amino-2-methylpropyl)(hydroxy)phosphinoyloxy)ethanoic acid;
2-(3-guanidinophenyl)-2-((1-(benzyloxycarbonyl)amino-3-methylbutyl)(hydroxy)phosphinoyloxy)ethanoic acid;
2-(2-chloro-3-guanidinophenyl)-2-((1-(benzyloxycarbonyl)amino-2-methylpropyl)(hydroxy)phosphinoyloxy)ethanoic acid;
2-(3-guanidinophenyl)-2-((1-(benzyloxycarbonyl)amino-1-phenylmethyl)(hydroxy)phosphinoyloxy)ethanoic acid;
2-(2-fluoro-3-guanidinophenyl)-2-((1-(benzyloxycarbonyl)amino-2-methylpropyl)(hydroxy)phosphinoyloxy)ethanoic acid;
2-(3-guanidinophenyl)-2-((1-(benzyloxycarbonyl)amino-1-cyclohexylmethyl)(hydroxy)phosphinoyloxy)ethanoic acid;
2-(2-methyl-3-guanidinophenyl)-2-((1-(benzyloxycarbonyl)amino-2-methylpropyl)(hydroxy)phosphinoyloxy)ethanoic acid;
2-(3-(amino)methylphenyl)-2-((1-(benzyloxycarbonyl)amino-2-methylpropyl)(hydroxy)phosphinoyloxy)ethanoic acid;
2-(3-(guanidinomethyl)phenyl)-2-((1-(benzyloxycarbonyl)amino-2-methylpropyl)(hydroxy)phosphinoyloxy)ethanoic acid;
2-(3-(1-iminoethylaminophenyl))-2-((1-(benzyloxycarbonyl)amino-2-methylpropyl)(hydroxy)phosphinoyloxy)ethanoic acid;
2-(3-(t-butoxycarbonylamino)methylphenyl)-2-((1-(benzyloxycarbonyl)amino-2-methylpropyl)(hydroxy)phosphinoyloxy)ethanoic acid;
2-(3-(ethoxycarbonylamino)methylphenyl)-2-((1-(benzyloxycarbonyl)amino-2-methylpropyl)(hydroxy)phosphinoyloxy)ethanoic acid;
2-(3-(isopropoxycarbonylamino)methylphenyl)-2-((1-(benzyloxycarbonyl)amino-2-methylpropyl)(hydroxy)phosphinoyloxy)ethanoic acid;
2-(3-(2,2-dimethylpropylcarbonylamino)methylphenyl)-2-((1-(benzyloxycarbonyl)amino-2-methylpropyl)(hydroxy)phosphinoyloxy)ethanoic acid;
2-(3-guanidinophenyl)-2-((1-(2-phenylethylcarbonyl)amino-2-methylpropyl)(hydroxy)phosphinoyloxy)ethanoic acid; and
2-(3-guanidinophenyl)-2-((1-(2-phenylethenylcarbonyl)amino-2-methylpropyl)(hydroxy)phosphinoyloxy)ethanoic acid.

36. The method of claim 33 wherein:
X is —O—;
R² is —P(O)(OR⁵)—R⁷—N(R⁵)—C(O)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂).

37. The method of claim 36 wherein the compound of formula (III) is selected from the group consisting of:
2-(3-guanidinophenyl)-2-[(1-(1-benzyloxycarbonylamino-2-(4-hydroxyphenyl)-ethylcarbonyl)amino-2-methylpropyl)(hydroxy)phosphinoyloxy]ethanoic acid;
2-(3-guanidinophenyl)-2-[(1-(1-benzyloxycarbonylamino-2-phenylethylcarbonyl)amino-2-methylpropyl)(hydroxy)phosphinoyloxy]ethanoic acid;
2-(2-fluoro-3-guanidinophenyl)-2-[(1-(1-benzyloxycarbonylamino-2-phenylethylcarbonyl)amino-2-methylpropyl)(hydroxy)phosphinoyloxy]ethanoic acid;
2-(3-guanidinophenyl)-2-[(1-(1-phenylcarbonylamino-2-phenylethylcarbonyl)amino-2-methylpropyl)(hydroxy)phosphinoyloxy]ethanoic acid;
2-(3-guanidinophenyl)-2-[(1-(1-ethoxycarbonylamino-2-phenylethylcarbonyl)amino-2-methylpropyl)(hydroxy)phosphinoyloxy]ethanoic acid;
2-(3-guanidinophenyl)-2-[(1-(1-benzyloxycarbonylamino-3-phenylpropylcarbonyl)amino-2-methylpropyl)(hydroxy)phosphinoyloxy]ethanoic acid; and
2-(3-(amino)methylphenyl)-2-[(1-(1-benzyloxycarbonylamino-3-phenylpropylcarbonyl)amino-2-methylpropyl)(hydroxy)phosphinoyloxy]ethanoic acid.

38. The method of claim 33 wherein:
X is —CH₂—;
R² is —P(O)(OR⁵)—R⁷—N(R⁵)—C(O)R⁶ or —P(O)(OR⁵)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂, —N(R⁵)—C(NR⁵)—N(R⁵)₂, —N(R⁵)—C(O)-N(R⁶)₂ and —N(R⁵)—C(O)—R⁷—N(R⁶₂).

39. The method of claim 38 wherein the compound of formula (III) is selected from the group consisting of:
2-(3-(amino)methylphenyl)-3-((1-(methylcarbonyl)amino-2-methylpropyl)(hydroxy)-phosphinoyl)propanoic acid;
2-(3-(hydrazinocarbonyl)phenyl)-3-((1-(benzyloxycarbonyl)amino-2-methylpropyl)-(hydroxy)phosphinoyl)propanoic acid;
2-(3-guanidinophenyl)-3-((1-(benzyloxycarbonyl)aminoethyl)(hydroxy)phosphinoyl)-propanoic acid;
2-(3-guanidinophenyl)-3-((1-(benzyloxycarbonyl)amino-3-methylbutyl)(hydroxy)phosphinoyl)propanoic acid;
2-(3-guanidinophenyl)-3-(((benzyloxycarbonyl)aminomethyl)(hydroxy)phosphinoyl)-propanoic acid;
2-(3-guanidinophenyl)-3-((1-(benzyloxycarbonyl)amino-2-methylpropyl)(hydroxy)-phosphinoyl)propanoic acid;
2-(2-chloro-5-guanidinophenyl)-3-((1-(benzyloxycarbonyl)amino-2-methylpropyl)(hydroxy)phosphinoyl)propanoic acid;
2-(3-(amino)methylphenyl)-3-((1-(benzyloxycarbonyl)amino-2-methylpropyl)(hydroxy)-phosphinoyl)propanoic acid; and
2-(3-(amino)methylphenyl)-3-((1-(2-phenylethylcarbonyl)amino-2-methylpropyl)-(hydroxy)phosphinoyl)propanoic acid.

40. The method of claim 33 wherein:
X is —CH₂—;
R² is —P(O)(OR⁵)—R⁷—N(R⁵)—C(O)—R⁷—N(R⁵)—C(O)OR⁸; and
R⁴ is aryl optionally substituted by one or more substituents selected from the group consisting of alkyl, halo, nitro, cyano, —N(R⁶)₂, —R⁷—N(R⁶)₂, —N(R⁶)—C(O)OR⁸, —R⁷—N(R⁶)—C(O)OR⁸, —N(R⁶)—C(O)—R⁶, —R⁷—N(R⁶)—C(O)—R⁶, —C(O)—N(R⁶)₂, —C(O)—N(R⁶)—N(R⁶)₂, —C(O)—R⁷—N(R⁶)₂, —N(R⁵)—C(NR⁵)—N(R⁵)₂, —N(R⁵)—C(O)—N(R⁶)₂ and —N(R⁵)—C(O)—R⁷—N(R⁶₂).

41. The method of claim 40 wherein the compound of formula (III) is selected from the group consisting of:
2-(3-guanidinophenyl)-3-(((1-benzyloxycarbonylamino-2-phenylethyl)carbonylaminomethyl)(hydroxy)phosphinoyl)propanoic acid;
2-(3-guanidinophenyl)-3-(((1-benzyloxycarbonylamino-2-phenylethyl)carbonylaminomethyl)(hydroxy)phosphinoyl)propanoic acid.

42. A method of treating pulmonary fibrosis comprising administering a therapeutically effective amount of a TAFI inhibitor to a patient in need thereof.
